(19) United States
(12) Patent Application Publication
(10) Pub. No.: US 2023/0190648 A1
(43) Pub. Date: Jun. 22, 2023

(54) Bioactive Tissue Derived Nanocomposite Hydrogels for Permanent Arterial Embolization and Enhanced Vascular Healing

(71) Applicant: Mayo Foundation for Medical Education and Research, Rochester, MN (US)

(72) Inventors: Rahmi Oklu, Chandler, AZ (US); Jingjie Hu, New York, NY (US)

(21) Appl. No.: 17/925,520

(22) PCT Filed: May 17, 2021
(86) PCT No.: PCT/US2021/032772

§ 371 Date: Nov. 15, 2022

Related U.S. Application Data
(60) Provisional application No. 63/025,705, filed on May 15, 2020.

(51) Int. Cl. A61K 9/06; A61K 47/46; A61K 47/02; A61K 9/00; A61K 49/04; A61L 24/00

(52) U.S. Cl. CPC A61K 9/06; A61K 9/0024; A61K 47/02; A61K 47/46; A61K 49/0438; A61L 24/001; A61L 24/0005; A61L 24/0031; A61L 24/0068; A61L 2300/44

(57) ABSTRACT

This document provides materials and methods for permanent arterial embolization and/or enhanced vascular healing. For example, materials and methods for using bioactive tissue derived nanocomposite hydrogels to enhance vascular healing are provided.

### CROSS-REFERENCE TO RELATED APPLICATIONS

This application claims the benefit of U.S. Pat. Application Serial No. 63/025,705, filed on May 15, 2020. The disclosure of the prior application is considered part of (and is incorporated by reference in) the disclosure of this application.

### STATEMENT AS TO FEDERALLY SPONSORED RESEARCH

This invention was made with government support under HL137193 awarded by National Institutes of Health. The government has certain rights in the invention.

### BACKGROUND

### 1. Technical Field

This document relates to materials and methods for permanent arterial embolization and/or enhanced vascular healing. For example, this document provides materials and methods for using bioactive tissue derived nanocomposite hydrogels to enhance vascular healing.

### 2. BACKGROUND INFORMATION

Transarterial embolization (TAE) is a minimally invasive procedure to selectively deliver embolic agents into arteries to occlude diseased or injured vasculature for therapeutic intent; it is considered as the first-line therapy for most gastrointestinal tract bleeding and unresectable hepatocellular tumors (Shin et al., Korean J. Radiol., 13:S31 (2012); Hu et al., Adv. Mater., 31:1901071 (2019); and Idee et al., Crit. Rev. Oncol. Hematol., 88:530 (2013)). Compared to invasive open surgeries, TAE offers a safe and efficient approach to control bleeding with better clinical outcomes and lower costs and has become a mainstay in treating many vascular diseases, including hemorrhage, aneurysms, vascular malformations, and hypervascular tumors (Hu et al., Adv. Mater., 31:1901071 (2019); Tarasconi et al., World J. Emerg. Surg., 14:3 (2019); Zhou et al., Sci. Rep., 6 (2016); Wang et al., Prog. Biomed. Eng., 2:012003 (2020); and Poursaid et al., J. Controlled Release, 240:414 (2016)). During TAE, a catheter is navigated through the vasculature under the guidance of X-ray fluoroscopy, and embolic agents are delivered to occlude the targeted vessels. A variety of embolic agents such as coils, beads, and liquid embolics are currently used in the clinic; however, their effectiveness is limited by high cost, recanalization, toxicity, risk of non-specific embolization and stroke (Lam et al., J. Neurosci. Meth., 329:108460 (2020); Zhu et al., Adv. Mater., 0:1805452 (2018); and Vaidya et al., Seminars in Interventional Radiology, 25:204 (2008)). In particular, the versatility of embolization poses a major challenge in developing embolic agents, primarily due to a wide range of target vessel sizes (from 5-10 µm diameter capillaries to 1-2 cm diameter arteries) and architecture of the blood vessels (e.g., aneurysms versus vascular malformations) to be embolized or the type of embolization required (e.g., permanent versus temporary) (Kessel and Ray (eds.), Techniques in Interventional Radiology, Transcatheter Embolization and Therapy, Springer-Verlag (2010)). For example, coils are commonly used solid embolic agents for the treatment of focal vascular conditions such as aneurysms and bleeding since they are intended to stay at the site of injury. However, liquid embolic agents such as Onyx (Medtronic, USA) are intended to travel distally from the site of release to penetrate finer vasculature, but they are associated with toxicity resulting from organic solvents (Hu et al., Adv. Mater., 31:1901071 (2019)). Moreover, the mismatch between the sizes of embolic agents and targeted vessels can give rise to numerous complications during embolization, such as non-target embolization, recurrent hemorrhage, and organ ischemia (Chuang et al., Am. J. Roentgenol., 137:809 (1981); and Tummala et al., Neurosurgery, 49:1059 (2001)). Therefore, it is of great importance to develop innovative strategies and new embolic agents with the ability to be adaptable to unique cases presented by each patient, possess desirable mechanical, radiopaque, and biological properties for effective embolization treatment, while also minimizing collateral injury to adjacent tissues (Hu et al., Adv. Mater., 31:1901071 (2019)).

### SUMMARY

To overcome the drawbacks of current embolic agents, strategic design of malleable hydrogels may offer significant advantages over both solid and liquid embolic agents used in the clinic today. Such hydrogels could be delivered using any catheter; they would form an impenetrable solid cast of various vessel geometries and sizes, avoiding embolization failure and recurrent bleeding, and provide the versatility that cannot be achieved by clinically used embolic agents. In addition, embolic agents used today merely lead to occlusion; next-generation embolic agents can also offer the flexibility to deliver therapeutics, including cells, drugs, gene therapy, and viral-vectors.

Injectable decellularized extracellular matrix (ECM) hydrogels provide an unparalleled therapeutic platform for minimally-invasive procedures in regenerative medicine and tissue engineering, such as tissue repair and organ replacement. Mainly comprised of polysaccharides, including glycosaminoglycans and hyaluronic acid and fibrous proteins including, collagen, elastin, fibronectin, and laminin, ECM offers a highly dynamic microenvironment providing biomechanical and biochemical cues for tissue morphogenesis and homeostasis. Decellularized ECMs from a variety of tissues have been used to make needle-based injectable hydrogels (Saldin et al., Acta Biomater., 49:1 (2017); Bejleri et al., Adv. Healthcare Mater., 8:1801217 (2019)); however, only porcine-derived cardiac ECM hydrogels obtained from the left ventricle of the heart has advanced into a clinical trial to treat myocardial infarction (Traverse et al., JACC: Basic to Translational Science, 4:659 (2019)). Studies have shown that cardiac ECM hydrogels can promote muscle regeneration, facilitate vascularization, and modulate macrophage polarization towards tissue healing without hemocompatibility issues in vivo (Traverse et al., JACC: Basic to Translational Science, 4:659 (2019); and Duran et al., Chapter 7 “Decellularized Extracellular Matrix: Characterization, Fabrication and Applications,” in The Royal Society of Chemistry, 2020). These properties suggest that cardiac ECM hydrogels are biocompatible and regenerative, which can be highly beneficial for the remodeling of embolized vessels where ingrowth of connective tissue is desired for permanent vessel occlusion. Furthermore, cardiac ECM hydrogel can undergo sol-gel transition at body temperature, and such a gelation effect may benefit embolotherapy as a result of improved gel stability at targeted sites (Saldin et al., Acta Biomater., 49:1 (2017)). It is also naturally shear-thinning, which is desired for transcatheter-based delivery (Saldin et al., Acta Biomater., 49:1 (2017)). Despite these advantages, injectable cardiac ECM hydrogel still suffers from poor mechanical properties (usually with modulus less than 10 Pa) and a rapid degradation profile (Saldin et al., Acta Biomater., 49:1 (2017)). These hinder its clinical value; the ability to withstand physiologic pressures inside an artery and remain structurally intact throughout the treatment period are critical for an effective embolic agent. Hence, the combination of tissue-derived ECM with synthetic components is required to generate biohybrid materials with strength and resistance to fragmentation while also maintaining ECM’s natural bioactivity (Bracaglia et al., Adv. Healthcare Mater., 4:2475 (2005)). To date, ECM based materials have not been investigated for embolotherapy in TAE, where catheters longer than 100 cm with inner diameter as small as 600 µm are commonly used.

As described herein, transcatheter embolization is a minimally invasive procedure that uses embolic agents to intentionally block diseased or injured blood vessels for therapeutic purposes. Embolic agents in clinical practice are limited by recanalization, risk of non-target embolization, failure in coagulopathic patients, high cost, and toxicity.

Here, a decellularized ECM based nanocomposite hydrogel was developed to provide superior mechanical stability, catheter injectability, retrievability, antibacterial properties and biological activity to prevent recanalization. The embolic efficacy of the shear-thinning ECM based hydrogel was shown in a porcine survival model of embolization in the iliac artery and the renal artery. ECM based hydrogel promotes arterial vessel wall remodeling and a fibroinflammatory response while undergoing significant biodegradation such that only 25% of the embolic material remains at 14 days. With its unprecedented pro-regenerative, antibacterial properties coupled with favorable mechanical properties, and its superior performance in anticoagulated blood, ECM based hydrogel has the potential to be the next generation biofunctional embolic agent that can successfully treat a wide range of vascular diseases.

In one aspect, this document features a composition comprising (or consisting essentially of or consisting of) a hydrogel described herein.

In another aspect, this document features a method for performing a permanent arterial embolization. The method comprises (or consists essentially of or consists of) administering a composition to a mammal to form a permanent arterial embolization. The composition comprises (or consists essentially of or consists of) a hydrogel described herein. The mammal can be a human.

In another aspect, this document features a method for enhancing vascular healing. The method comprises (or consists essentially of or consists of) administering a composition to a mammal to enhance vascular healing. The composition comprises (or consists essentially of or consists of) a hydrogel described herein. The mammal can be a human.

In general, one aspect of this document features hydrogel compositions including ECM and a nanoclay material. The hydrogel composition can include about 1 wt% of the decellularized ECM. The hydrogel composition can include from about 1 wt% to about 5.5 wt% of the nanoclay material. The hydrogel composition can include about 4.5 wt% of the nanoclay material. The nanoclay material can be a silicate nanoclay. The hydrogel composition also can include a radiopaque contrast agent. The hydrogel composition can include from about 18 wt% to about 27 wt% radiopaque contrast agent. The hydrogel composition can include from about 27 wt% of the radiopaque contrast agent. The radiopaque contrast agent can be iohexol, tantalum microparticles, iodized oil, or iodixanol.

In another aspect, this document features methods for embolization of a blood vessel within a mammal. The methods can include, or consist essentially of, administering, to a blood vessel within a mammal, a hydrogel composition including decellularized ECM and a nanoclay material. The mammal can be a human. The administering can include catheter-directed administration. The administering can include administering from about 1 cc to about 3 cc of the hydrogel composition.

In another aspect, this document features methods for enhancing vascular healing of a blood vessel within a mammal. The methods can include, or consist essentially of, administering, to a blood vessel within a mammal, a hydrogel composition comprising decellularized ECM and a nanoclay material. The mammal can be a human. The administering can include catheter-directed administration. The administering can include administering from about 1 cc to about 3 cc of the hydrogel composition.

In another aspect, this document features methods for reducing blood flow in a blood vessel within a mammal. The methods can include, or consist essentially of, administering, to a blood vessel within a mammal, a hydrogel composition comprising decellularized ECM and a nanoclay material. The mammal can be a human. The administering can include catheter-directed administration. The administering can include administering from about 1 cc to about 3 cc of the hydrogel composition.

In another aspect, this document features methods for inducing collagen deposition within a mammal. The methods can include, or consist essentially of, administering, to a mammal, a hydrogel composition including decellularized ECM and a nanoclay material, where the hydrogel composition is effective to induce collagen deposition at the delivery site. The mammal can be a human. The administering can include catheter-directed administration. The administering can include administering from about 1 cc to about 3 cc of the hydrogel composition.

In another aspect, this document features methods for inducing angiogenesis within a mammal. The methods can include, or consist essentially of, administering, to a mammal, a hydrogel composition including decellularized ECM and a nanoclay material, where the hydrogel composition is effective to induce angiogenesis at the delivery site. The mammal can be a human. The administering can include catheter-directed administration. The administering can include administering from about 1 cc to about 3 cc of the hydrogel composition.

In another aspect, this document features methods for inducing cellular proliferation within a mammal. The methods can include, or consist essentially of, administering, to a mammal, a hydrogel composition including decellularized ECM and a nanoclay material, where the hydrogel composition is effective to induce cellular proliferation at the delivery site. The mammal can be a human. The administering can include catheter-directed administration. The administering can include administering from about 1 cc to about 3 cc of the hydrogel composition.

In another aspect, this document features methods for treating a mammal having a bleeding disorder. The methods can include, or consist essentially of, administering, to a mammal having a bleeding disorder, a hydrogel composition comprising decellularized ECM and a nanoclay material. The bleeding disorder can be a non-traumatic hemorrhage, a traumatic hemorrhage, a ruptured aneurysm, a saccular aneurysm, a vascular malformation, or an endoleak. The mammal can be a human. The administering can include catheter-directed administration. The administering can include administering from about 1 cc to about 3 cc of the hydrogel composition.

In another aspect, this document features methods for treating a mammal having a tumor. The methods can include, or consist essentially of, administering, to a blood vessel within a mammal having a tumor that is feeding the tumor, a hydrogel composition including decellularized ECM and a nanoclay material. The tumor can be a benign tumor. The tumor can be a malignant tumor. The tumor can be a hepatic tumor, a uterine fibroid, or a prostate tumor. The mammal can be a human. The administering can include catheter-directed administration. The administering can include administering from about 1 cc to about 3 cc of the hydrogel composition.

Unless otherwise defined, all technical and scientific terms used herein have the same meaning as commonly understood by one of ordinary skill in the art to which this disclosure pertains. Methods and materials are described herein for use in the present disclosure; other, suitable methods and materials known in the art can also be used. The materials, methods, and examples are illustrative only and not intended to be limiting. All publications, patent applications, patents, sequences, database entries, and other references mentioned herein are incorporated by reference in their entirety. In case of conflict, the present specification, including definitions, will control.

The details of one or more embodiments of the invention are set forth in the accompanying drawings and the description below. Other features, objects, and advantages of the invention will be apparent from the description and drawings, and from the claims.

### DESCRIPTION OF DRAWINGS

FIG. 1. Schematics of ECM-NC nanocomposite gel fabrication and in vivo embolization testing. The black dotted line outlined the left ventricle of a porcine heart for decellularization.

FIGS. 2A-2J. ECM preparation and characterization. FIG. 2A) Decellularized cardiac tissue preparation comprised of dissecting the left ventricle of the porcine heart, followed by decellularization and lyophilization. FIG. 2B) Representative images of cardiac tissue characterization pre and post decellularization including H&E, immunostaining of collagen-I, fibronectin and laminin, and SEM. FIG. 2C) Decellularized cardiac tissue being digested and neutralized to form ECM solution, which underwent sol-gel transition at 37° C. creating a nano-fibered mesh. FIG. 2D) dsDNA amount in the native tissue and ECM, confirming successful decellularization. FIG. 2E) FTIR spectra of ECM samples prepared from three different pigs, showing composition consistency. FIG. 2F) SDS-PAGE gel of ECM samples prepared from different porcine hearts (ECM1-ECM3) individually, pooled samples, and rat tail collagen-I protein (Col-I), showing consistency of protein composition in prepared ECMs with the major component being Col-I. FIG. 2G) Representative turbidimetric gelation kinetics of ECMs at concentrations of 9, 12, and 20 mg/mL. FIG. 2H) Gelation kinetics of ECMs at 37° C. measured by rheometry, showing concentration-dependent kinetics. FIG. 2I) Representative G′ and G″ curves as a function of the amplitude of oscillatory shear strain measured for ECMs. FIG. 2J) Shear rate sweep revealing the shear-thinning nature of ECM (20 mg/mL) at 4° C. and 37° C. ****p < 0.0001. Each data point represents average ± standard deviation.

FIGS. 3A-3L. Mechanical properties and biofunctionalities of ECM-NC nanocomposite hydrogels. Rheology of xECM4.5NC, as characterized by (FIG. 3A) Shear rate sweeps. FIG. 3B) G′ measured from oscillatory strain sweeps performed at 10 rad/second (n=3). FIG. 3C) Yield stress calculated from oscillatory strain sweeps (n=3). FIG. 3D) Oscillatory frequency sweeps performed at 0.1 % strain. FIG. 3E) Time sweep revealing recoverability of gels under alternating cycles between 2-minute low 0.1 % strain and 1-minute high 100 % strain at 10 rad/second. FIG. 3F) Representative injection force curves, showing breakloose and injection forces. FIG. 3G) Cell viability of L-929 fibroblasts seeded directly on ECM gels 16 hours, showing enhanced cell viability with an increasing amount of ECM in the gels (n=8). FIG. 3H) FTIR spectra of ECM, NC, EMH, and EMH-I, showing chemical composition. FIG. 3I) EMH-I extruded from a 2.8 F catheter by manual injection and schematics showing interactions between ECM proteins, NC and iohexol network under shear. FIG. 3J) The pressure required to displace control (PBS alone), NC, EMH, and EMH-I (n=3). Inset shows the schematics of tube-based setup for in vitro occlusion assessment. FIG. 3K) Viability of L-929 cells after incubated with ECM, NC, EMH, and EMH-I extractions at different concentrations for 24 hours, showing no toxicity of the gels (n=12). FIG. 3L) Antibacterial properties tested by measuring the optical density of E. coli suspensions that were cultured on EMH and EMH-I (n=8). ns, not significant; *p < 0.05, **p < 0.01, ****p < 0.0001. Each data point represents average ± standard deviation.

FIGS. 4A-4J. Histological analysis of the gel specimens and surrounding tissues taken at different time points in rat subcutaneous injections. FIG. 4A) Representative H&E stain of explanted NC, EMH and EMH-I at D3, D14, and D28 post subcutaneous injection. The total amount of infiltrated cells inside of the implanted region at D3 (FIG. 4B), D14 (FIG. 4C), and D28 (FIG. 4D). (n=4). FIG. 4E) Representative Masson’s Trichrome staining at 28 days. Black arrows show the fibrotic capsule. FIG. 4F) Measured capsule thickness at D28 (n=8). Reduced capsule thickness formed around EMH and EMH-I compared to NC indicated better biocompatibility. FIG. 4G) Representative MPO immunostaining at D3. Black arrows point towards MPO positive cells. FIG. 4H) Total amount of MPO in the implant area at D3, D14, and D28 showing subsided inflammatory responses of EMH and EMH-I over the long term (n=6). FIG. 4I) Representative CD31 immunostaining at the gel-tissue interface at D28. Black arrows point towards CD31 positive blood vessels. FIG. 4J) Amount of vessels at the dermis and subcutaneous sites at D28 showing pro-angiogenesis properties of EMH and EMH-I (n=4). The asterisks indicate the location of the injected material. One-way ANOVA tests were performed for statistical analysis. ns, not significant; *p < 0.05, **p < 0.01, ****p < 0.0001. Each data point represents average ± standard error.

FIGS. 5A-5K. Arterial embolization in a porcine model. Digitally subtracted angiography (DSA) of internal iliac artery (IIA) before (FIG. 5A; arrow pointing the patent IIA), and after embolization (FIG. 5B; arrow pointing embolized IIA with no flow). FIG. 5C) Fluoroscopic image of EMH-I occluding IIA, showing its radiopacity and visibility (black arrow). FIG. 5D) Reconstructed 3D CTA image showing occluded IIA at D14. IIA is missing from 3D CTA since it is embolized and does not enhance (IIA outlined by black dot and pointed with black arrow). FIG. 5E) Micro-CT images, both sagittal and transverse sectional views, of embolized IIA at D0 and D14. FIG. 5F) Immunostaining of collagen-I, fibronectin, and laminin on embolized IIA at D0. Representative images of H&E, elastin, trichrome and PCNA staining of embolized IIA at D0 (FIG. 5G), and D14 (FIG. 5H) are shown. FIG. 5I) Quantitative analysis of internal elastic lamina (n=4) showing reduction at D14. FIG. 5J) PCNA positive cells in the lumen of the embolized artery were counted, showing a significant amount of proliferating cells in the lumen at D14 (n=4). FIG. 5K) In vivo degradation profile of EMH-I volume inside of IIA obtained from microCT analysis. Bar scales for FIG. 5E and FIG. 5F are 1 mm, for full views in FIG. 5G and FIG. 5H are 1 mm, for interface and center images in FIG. 5G and FIG. 5H are 150 µm. ***p < 0.005, ****p < 0.0001. Each data point represents average ± standard error.

FIGS. 6A-6I. Renal artery embolization in a porcine model. DSA of the left kidney before (FIG. 6A; black arrow pointing the main renal artery being patent), and after the delivery of EMH-I (FIG. 6B; black arrow pointing the main renal artery being occluded). FIG. 6C) Fluoroscopic image showing radiopaque EMH-I blocking the main renal artery as well as segmental arterial branches (black arrow) in kidney. FIG. 6D) 3D CTA image of non-embolized and embolized kidneys 14 days post-procedure. Embolized kidney is missing (location denoted by white arrow) due to the absence of blood flow and non-enhancement. FIG. 6E) Axial CTA image showing the non-enhancing parenchyma of the embolized kidney (white dotted outline) compared to the control (orange dotted outline). FIG. 6F) Volume of embolized kidney and non-treated kidney as determined by CTA (n=4). FIGS. 6G and 6H) Gross image of excised kidneys, and representative H&E images of embolized kidney showing EMH-I (asterisks) in embolized vessels and the renal cortex at D0, and fibrosis with loss of architecture at D14. FIG. 6I) CT images of normal organs in animals that received EMH-I. Lung, liver, spleen, heart, and brain are outlined by dotted line. White arrows point to widely patent vessels in hind limbs showing no evidence for non-target embolization. *p < 0.05. Each data point represents average ± standard error.

FIGS. 7A-7E. FIG. 7A) Optical density of ECM solutions measured at 405 nm during isothermal gelation at 37° C. FIG. 7B) Fitting of the turbidimetric gelation curve to calculate t_{½}, t_{lag} and S. Summary of t_{½} (FIG. 7C), t_{lag} (FIG. 7D), and S (FIG. 7E) of gelation kinetics for ECM of 9, 12 and 20 mg/mL (n=3). ns, not significant; *p < 0.05, **p < 0.01, ****p < 0.0001. Each data point represents average ± standard error.

FIG. 8. Hydrodynamic diameter of NC particles in water measured by dynamic light scattering.

FIGS. 9A-9C. FIG. 9A) Representative amplitude sweeps of the xECM4.5NC gels, showing G′ and G″ curves as function of the oscillatory shear strain. Representative schematics showing the calculation procedure of critical strain γ_{c} extracted from G′ curve (FIG. 9B), and yield stress σ_{y} extrapolated from stress-strain curve (FIG. 9C), for each gel.

FIG. 10. Summary of break loose and injection forces of xECM4.5NC nanocomposite gels. The forces suggest the comfortable delivery of the xECM4.5NC gels through 2.8 F 110 cm catheter by manual injection.

FIGS. 11A-11F. Rheology of ECM-NC gels with 5.5 wt % of total solid content. FIG. 11A) Time-dependent plots of shear stress versus shear rate. FIG. 11B) Shear-rate sweeps, showing shear-thinning properties. FIG. 11C) Amplitude sweeps performed at 10 rad/second. FIG. 11D) Summary of G′ at 25° C. and 37° C. (n=3). G′ increased with increased ECM amount and decreased NC content. FIG. 11E) Oscillatory frequency sweeps performed at 10 rad/second at 0.1 % strain. FIG. 1 1F) Thixotropy test, showing recoverability of the gels. Deformation and recovery of gels evolved over time from repeated cycles of 2-minute low 0.1 % strain and 1-minute high 100 % strain oscillations at 10 rad/second.

FIGS. 12A-12B. FIG. 12A) Representative injection curve of ECM-NC gels with a constant total solid amount of 5.5 wt %. FIG. 12B) Summary of break loose force and injection force (n=5). The forces indicate the comfortable delivery of the formulated gels through a 2.8 F 110 cm catheter by manual injection.

FIGS. 13A-13F. Rheological properties of radiopaque xECM4.5NC-I gels. FIG. 13A) Time-dependent plots of shear stress versus shear rate. FIG. 13B) Shear-rate sweeps, showing shear-thinning properties. FIG. 13C) Amplitude sweeps performed at 10 rad/second. FIG. 13D) Summary of G′ at 25° C. and 37° C. (n=3). G′ increased with increased ECM amount at constant NC content. FIG. 13E) Oscillatory frequency sweeps performed at 10 rad/second at 0.1% strain. FIG. 13F) Thixotropy test, showing recoverability of radiopaque xECM4.5NC-I gels. Deformation and recovery of gels evolved over time from repeated cycles of 2-minute low 0.1 % strain and 1-minute high 100 % strain oscillations at 10 rad/second.

FIGS. 14A-14B. FIG. 14A) Representative injection curves of radiopaque xECM4.5NC-I gels. FIG. 14B) Summary of break loose force and injection forces (n=5). The forces reveal the comfortable delivery of the xECM4.5NC-I gels through 2.8 F 110 cm catheter by manual injection.

FIG. 15. Representative SEM images of NC, EMH, and EMH-I at high and low magnifications. NC had a relatively large flaky, and non-connective structure. When ECM was added into NC, the formed EMH appeared to be compact with struts that connected the adjacent structures. With the addition of iohexol into EMH, formulated EMH-I showed a porous, dense, and organized structure, which gave rise to its enhanced mechanical property.

FIG. 16. Representative pressure displacement curves of PBS (control), NC, EMH, and EMH-I. The peak force corresponded to the maximum pressure each material can withstand at a flow rate of 50 mL/minute. EMH-I showed the highest pressure, followed by EMH and NC. The dotted line represented the physiological pressure of 16 kPa, equivalent to 120 mmHg.

FIG. 17. Graphic summary of CBC parameters of subcutaneously injected rats at D0, D3, D14, and D28. *p < 0.05. Rats were healthy, and no infection was observed. Each data point represents the average ± standard error (n=4).

FIGS. 18A-18B. Representative histological images of subcutaneously injected NC, EMH, EMH-I, and control (saline injection) in rats 3, 14, and 28 days post-injection. H&E staining (FIG. 18A); and Masson’s Trichrome staining (FIG. 18B) show a downward trend in the cross-sectional area of explanted NC, EMH, and EMH-I over 28 days. Note that the fracturing of the dermis, with increased clear space, is due to processing artifact, as reviewed by a board-certified pathologist. Bar scales for all images are 3 mm.

FIG. 19. The cross-sectional area of explanted NC, EMH, and EMH-I at 3, 14, and 28 days post subcutaneous injection (n=4). *p < 0.05. All materials underwent gradual in vivo degradation. Each data point represents the average ± standard error.

FIG. 20. Representative immunohistochemistry images of EMH-I in embolized iliac artery focusing at vessel/EMH-I interface and vessel center for collagen-I, fibronectin, and laminin at D0. These images confirmed the preservation of critical ECM proteins in EMH-I gels when used for embolization.

FIG. 21. Arterial embolization showing baseline DSA, post-embolization DSA, and fluoroscopic image of EMH-I in IIAs of four pigs, P1, P2, P3, and P4, showing complete occlusion and successful embolization in all animals. Fluoroscopic images demonstrate the embolized IIA because the EMH-I contains iohexol. Black arrows represent the IIA.

FIG. 22. CTA and 3D CTA pelvis images acquired 14 days post arterial embolization, showing complete occlusion of IIA for all 4 animals. Orange arrows indicate the embolized artery in CTA, and black arrows indicate the embolized artery not visualized in the 3D CTA due to lack of blood flow from embolization.

FIG. 23. Porcine renal artery embolization showing baseline DSA, post-embolization DSA, and fluoroscopic image of EMH-I in renal vasculatures for four pigs, P1, P2, P3, and P4. The missing renal vasculature after EMH-I infusion confirmed successful embolization in all four pigs. Fluoroscopic images demonstrate the EMH-I within the artery; they are visible under x-ray because they contain iohexol. Black arrows indicate the main renal artery.

FIG. 24. CTA images acquired 14 days post renal artery embolization. The embolized kidney (white dotted outline) demonstrates absence of enhancement and appears smaller in size compared to contralateral normal kidney in all four animals. Corresponding 3D CTA images indicate absence of the embolized kidneys (white arrows), confirming successful renal artery embolization.

FIG. 25. Representative microCT images of embolized kidneys collected at day 0 and day 14 after EMH-I embolization. H&E staining of the renal artery (location marked by white line) showed occlusion at D0 and persistent occlusion with evidence for remodeling of the renal artery at D14. D14 sample shows circumferential degradation of the biomaterial and connective tissue deposition with residual EMH-I centrally.

FIG. 26. CT images acquired 14 days post embolization in the porcine model, showing normal findings with preserved hindlimb perfusion in all four animals. Lung, liver, spleen, heart, and brain are outlined by dotted line. White arrows indicate widely patent distal hindlimb vessels.

### DETAILED DESCRIPTION

This document relates to materials and methods for permanent arterial embolization and/or enhanced vascular healing. For example, this document provides materials and methods for using bioactive tissue derived nanocomposite hydrogels to enhance vascular healing. In some cases, one or more hydrogel compositions provided herein can be delivered to one or more blood vessels within a mammal (e.g., a human) to induce formation of a thrombus (e.g., an artificial embolus) within the blood vessel(s). In some cases, one or more hydrogel compositions provided herein can be delivered to one or more blood vessels within a mammal (e.g., a human) to form an embolus (e.g., an artificial embolus) within the blood vessel(s).

A hydrogel composition provided herein can include decellularized ECM and one or more nanoclay materials. In some cases, a hydrogel composition provided herein can be sterile. In some cases, a hydrogel composition provided herein can be anti-bacterial. In some cases, a hydrogel composition provided herein can be bioactive. For example, a hydrogel composition provided herein can be designed to include one or more therapeutic agents.

A hydrogel composition provided herein (e.g., a hydrogel composition including decellularized ECM and one or more nanoclay materials) can include any amount of decellularized ECM. For example, a hydrogel composition provided herein can include from about 0% (wt%) to about 1% (wt%) decellularized ECM. In some cases, a hydrogel composition provided herein can include about 1% (wt%) decellularized ECM. For example, a hydrogel composition provided herein can include from about 0 mg/mL to about 12 mg/mL (e.g., from about 0 mg/mL to about 11 mg/mL, from about 0 mg/mL to about 10 mg/mL, from about 0 mg/mL to about 7 mg/mL, from about 0 mg/mL to about 5 mg/mL, from about 0 mg/mL to about 3 mg/mL, from about 1 mg/mL to about 12 mg/mL, from about 2 mg/mL to about 12 mg/mL, from about 3 mg/mL to about 12 mg/mL, from about 4 mg/mL to about 12 mg/mL, from about 5 mg/mL to about 12 mg/mL, from about 8 mg/mL to about 12 mg/mL, from about 10 mg/mL to about 12 mg/mL, from about 1 mg/mL to about 10 mg/mL, from about 3 mg/mL to about 8 mg/mL, from about 5 mg/mL to about 6 mg/mL, from about 1 mg/mL to about 4 mg/mL, or from about 4 mg/mL to about 8 mg/mL) decellularized ECM. In some cases, a hydrogel composition provided herein can include about 12 mg/mL decellularized ECM.

A hydrogel composition provided herein (e.g., a hydrogel composition including decellularized ECM and one or more nanoclay materials) can include any type of decellularized ECM. Decellularized ECM can be obtained using any appropriate method. Methods for obtaining decellularized ECM can be performed as described in, for example, Example 1. In some cases, decellularized ECM can be obtained as described elsewhere (see, e.g.,Wolf et al., Biomaterials, 33(29):7028-7038 (2012); Gilpin et al., Biomed. Res. Int., 2017:9831534 (2017); Faulk et al., J. Clin. Exp. Hepatol., 5(1):69-80 (2015); and Saldin et al., Acta Biomaterialia, 49:1-15 (2017). In some cases, decellularized ECM can be lyophilized. Decellularized ECM can include any ECM components. Examples of ECM components that can be present in decellularized ECM include, without limitation, collagen-I polypeptides, fibronectin polypeptides, laminin polypeptides, collagen-III polypeptides, collagen-IV polypeptides, and sulfated glycosaminoglycans (sGAGs). Decellularized ECM can include any amount of cellular remnants (e.g., DNA). In some cases, decellularized ECM can have a DNA content of less than 50 ng of DNA per mg tissue (ng/mg). For example, decellularized ECM can have a DNA content of from about 0 ng/mg to about 50 ng/mg (e.g., from about 0 ng/mg to about 40 ng/mg, from about 0 ng/mg to about 30 ng/mg, from about 0 ng/mg to about 20 ng/mg, from about 0 ng/mg to about 10 ng/mg, from about 10 ng/mg to about 50 ng/mg, from about 20 ng/mg to about 50 ng/mg, from about 30 ng/mg to about 50 ng/mg, from about 40 ng/mg to about 50 ng/mg, from about 10 ng/mg to about 40 ng/mg, from about 20 ng/mg to about 30 ng/mg, from about 10 ng/mg to about 20 ng/mg, from about 2 ng/mg to about 30 ng/mg, or from about 3 ng/mg to about 40 ng/mg). In some cases, decellularized ECM can include DNA fragments that are less than 200 base pairs (bp; e.g., less than 175 bp, less than 150 bp, less than 125 bp, less than 100 bp, less than 75 bp, less than 50 bp, or less than 25 bp) in length.

A hydrogel composition provided herein (e.g., a hydrogel composition including decellularized ECM and one or more nanoclay materials) can include any appropriate nanoclay material. In some cases, a hydrogel composition can include a single type of nanoclay material. In some cases, a hydrogel composition can include two or more (e.g., two, three, four, or more) types of nanoclay materials. A nanoclay material that can be included in a hydrogel composition can be in any appropriate form. For example, a nanoclay material can be a powder. In some cases, a nanoclay material can be swellable (e.g., a nanoclay material that swells to produce a gel such as a hydrogel when dispersed in a liquid such as water). Examples of nanoclay materials that can be included in a hydrogel composition provided herein include, without limitation, silicate nanoclay (e.g., a phyllosilicate nanoclay such as Laponite®), montmorillonite, sepiolite, and kaolinite.

A hydrogel composition provided herein (e.g., a hydrogel composition including decellularized ECM and one or more nanoclay materials) can include any amount of one or more nanoclay materials. For example, a hydrogel composition provided herein can include from about 1% (wt%) to about 5.5% (wt%) (e.g., from about 1% to about 4%, from about 1% to about 3%, from about 1% to about 2%, from about 2% to about 5%, from about 3% to about 5%, from about 4% to about 5%, from about 2% to about 4%, from about 1% to about 2%, from about 2% to about 3%, or from about 3% to about 4%) nanoclay material(s). In some cases, a hydrogel composition provided herein can include about 4.5% (wt%) nanoclay material(s) (e.g., Laponite®). For example, a hydrogel composition provided herein can include from about 1 mg/mL to about 45 mg/mL (e.g., from about 1 mg/mL to about 40 mg/mL, from about 1 mg/mL to about 30 mg/mL, from about 1 mg/mL to about 20 mg/mL, from about 1 mg/mL to about 10 mg/mL, from about 10 mg/mL to about 45 mg/mL, from about 20 mg/mL to about 45 mg/mL, from about 30 mg/mL to about 45 mg/mL, from about 40 mg/mL to about 45 mg/mL, from about 10 mg/mL to about 40 mg/mL, from about 20 mg/mL to about 30 mg/mL, from about 10 mg/mL to about 20 mg/mL, from about 20 mg/mL to about 30 mg/mL, or from about 30 mg/mL to about 40 mg/mL) nanoclay material(s). In some cases, a hydrogel composition provided herein can include about 45 mg/mL nanoclay material(s) (e.g., Laponite^{®}).

A hydrogel composition provided herein (e.g., a hydrogel composition including decellularized ECM and one or more nanoclay materials) can have any ratio of decellularized ECM to nanoclay materials. For example, a hydrogel composition provided herein can have a ratio of decellularized ECM to nanoclay materials of from about 0:4.5 to about 1:4.5. In some cases, a hydrogel composition provided herein can have a ratio of decellularized ECM to nanoclay materials of about 1:4.5.

A hydrogel composition provided herein (e.g., a hydrogel composition including decellularized ECM and one or more nanoclay materials) can have any amount of decellularized ECM and nanoclay materials. For example, a hydrogel composition provided herein can have from about 1% (wt%) to about 5.5% (wt%) decellularized ECM and nanoclay materials. In some cases, a hydrogel composition provided herein can have a total amount of decellularized ECM and nanoclay materials of about 5.5 % (wt%).

In some cases, a hydrogel composition provided herein (e.g., a hydrogel composition including decellularized ECM and one or more nanoclay materials) can include one or more contrast agents. For example, a hydrogel composition provided herein can be designed to include one or more radiopaque contrast agents. In some cases, a hydrogel composition can include a single type of radiopaque contrast agent. In some cases, a hydrogel composition can include two or more (e.g., two, three, four, or more) types of radiopaque contrast agents. Examples of radiopaque contrast agents that can be included in a hydrogel composition provided herein include, without limitation, iohexol, tantalum microparticles, iodized oil, and iodixanol.

A hydrogel composition provided herein (e.g., a hydrogel composition including decellularized ECM and one or more nanoclay materials) can include any amount of radiopaque contrast agent(s). For example, a hydrogel composition provided herein can include from about 0% (wt%) to about 27% (wt%) radiopaque contrast agent(s). In some cases, a hydrogel composition provided herein can include about 27% (wt%) radiopaque contrast agent(s) (e.g., iohexol) (e.g., iohexol). For example, a hydrogel composition provided herein can include from about 0 mg/mL to about 270 mg/mL (e.g., from about 0 mg/mL to about 250 mg/mL, from about 0 mg/mL to about 200 mg/mL, from about 0 mg/mL to about 150 mg/mL, from about 0 mg/mL to about 100 mg/mL, from about 0 mg/mL to about 50 mg/mL, from about 50 mg/mL to about 270 mg/mL, from about 100 mg/mL to about 270 mg/mL, from about 150 mg/mL to about 270 mg/mL, from about 200 mg/mL to about 270 mg/mL, from about 250 mg/mL to about 270 mg/mL, from about 50 mg/mL to about 250 mg/mL, from about 100 mg/mL to about 200 mg/mL, from about 50 mg/mL to about 150 mg/mL, or from about 150 mg/mL to about 250 mg/mL) radiopaque contrast agent(s). In some cases, a hydrogel composition provided herein can include about 270 mg/mL radiopaque contrast agent(s) (e.g., iohexol).

When a hydrogel composition provided herein (e.g., a hydrogel composition including decellularized ECM and one or more nanoclay materials) includes one or more radiopaque contrast agents, the hydrogel composition can be visualized (e.g., within a mammal) using any appropriate method. For example, imaging techniques such as ultrasound, computed tomography, magnetic resonance imaging, and/or fluoroscopy can be used to visualize a hydrogel composition provided herein.

In some cases, a hydrogel composition provided herein (e.g., a hydrogel composition including decellularized ECM and one or more nanoclay materials) can include about 1 wt% decellularized ECM and about 4.5 wt% nanoclay material(s). For example, a hydrogel composition provided herein can include about 1 wt% decellularized ECM, about 4.5 wt% nanoclay material(s), and about 27 wt% iohexol.

In some cases, a hydrogel composition provided herein (e.g., a hydrogel composition including decellularized ECM and one or more nanoclay materials) can be biodegradable (e.g., can biodegrade within a mammal). For example, a volume of a hydrogel composition delivered to a blood vessel within a mammal (e.g., a human) can decrease over time. In some cases, a volume of a hydrogel composition delivered to a blood vessel within a mammal (e.g., a human) can decrease by at least about 25% (e.g., at least about 30%, at least about 40%, at least about 50%, at least about 60%, at least about 70%, or at least about 75%) over time. In some cases, a volume of a hydrogel composition delivered to a blood vessel within a mammal (e.g., a human) can decrease for about 28 days following delivery. For example, a volume of a hydrogel composition delivered to a blood vessel within a mammal (e.g., a human) can decrease by at least about 50% (e.g., at least 75%) for about 28 days following delivery.

In some cases, when a hydrogel composition provided herein (e.g., a hydrogel composition including decellularized ECM and one or more nanoclay materials) biodegrades after being delivered to a blood vessel within a mammal (e.g., a human), the biodegraded hydrogel composition can be replaced with fibrotic tissue (e.g., permanent fibrotic tissue).

In some cases, a hydrogel composition provided herein (e.g., a hydrogel composition including decellularized ECM and one or more nanoclay materials) can be a shear-thinning composition. For example, a viscosity of a hydrogel composition provided herein can decrease under a shear rate of from about 0.001 1/second to about 10001/second (e.g., from about 0.001 1/second to about 7001/second, from about 0.001 1/second to about 5001/second, from about 0.001 1/second to about 300 1/second, from about 0.001 1/second to about 2001/second, from about 0.001 1/second to about 1001/second, from about 0.1 1/second to about 10001/second, from about 11/second to about 10001/second, from about 1001/second to about 10001/second, from about 3001/second to about 10001/second, from about 500 1/second to about 10001/second, from about 8001/second to about 10001/second, from about 0.1 1/second to about 8001/second, from about 11/second to about 500 1/second, from about 1001/second to about 300⅟second, from about 11/second to about 2001/second, from about 2001/second to about 4001/second, from about 400 1/second to about 6001/second, or from about 6001/second to about 8001second). In some cases, a viscosity of a hydrogel composition provided herein can decrease under a shear rate of about 1001/second.

In some cases, a hydrogel composition provided herein (e.g., a hydrogel composition including decellularized ECM and one or more nanoclay materials) can have a displacement pressure that is higher than the mean pressure of a blood vessel (e.g., a healthy blood vessel). For example, a hydrogel composition provided herein can have a displacement pressure of from about 65 kPa to about 119 kPa (e.g., from about 65 kPa to about 100 kPa, from about 65 kPa to about 80 kPa, from about 75 kPa to about 119 kPa, from about 100 kPa to about 119 kPa, from about 70 kPa to about 110 kPa, from about 80 kPa to about 100 kPa, from about 65 kPa to about 85 kPa, or from about 85 kPa to about 105 kPa). For example, a hydrogel composition provided herein can have a displacement pressure of from about 487 mm Hg to about 892 mm Hg (e.g., from about 487 mm Hg to about 800 mm Hg, from about 487 mm Hg to about 700 mm Hg, from about 487 mm Hg to about 600 mm Hg, from about 487 mm Hg to about 500 mm Hg, from about 500 mm Hg to about 892 mm Hg, from about 600 mm Hg to about 892 mm Hg, from about 700 mm Hg to about 892 mm Hg, from about 800 mm Hg to about 892 mm Hg, from about 500 mm Hg to about 800 mm Hg, from about 600 mm Hg to about 700 mm Hg, from about 500 mm Hg to about 600 mm Hg, from about 600 mm Hg to about 700 mm Hg, or from about 800 mm Hg to about 800 mm Hg).

In some cases, a hydrogel composition provided herein (e.g., a hydrogel composition including decellularized ECM and one or more nanoclay materials) can be shelf stable (e.g., does not separate and/or degrade during storage). For example, a hydrogel composition provided herein can be stable (e.g., without phase separation) for from about 0 hours to about 12 months (e.g., from about 0 hours to about 9 months, from about 0 hours to about 6 months, from about 0 hours to about 3 months, from about 2 hours to about 12 months, from about 6 hours to about 12 months, from about 12 hours to about 12 months, from about 24 hours to about 12 months, from about 48 hours to about 12 months, from about 36 hours to about 12 months, from about 48 hours to about 12 months, from about 1 month to about 12 months, from about 3 months to about 12 months, from about 6 months to about 12 months, from about 9 months to about 12 months, from about 2 hours to about 9 months, from about 6 hours to about 6 months, from about 12 hours to about 3 months, from about 24 hours to about 2 months, from about 36 hours to about 1 month, from about 2 hours to about 12 hours, from about 12 hours to about 36 hours, from about 36 hours to about 72 months, from about 1 month to about 3 months, from about 3 months to about 6 months, or from about 6 months to about 9 months).

A hydrogel composition provided herein (e.g., a hydrogel composition including decellularized ECM and one or more nanoclay materials) can be made using any appropriate method. When a hydrogel composition provided herein also includes one or more contrast agents, the decellularized ECM and one or more nanoclay materials can be mixed first, and then one or more contrast agents can be added. For example, centrifugal mixing, vortexing, and/or planetary mixing can be used for mixing (e.g., homogenous mixing) of decellularized ECM and one or more nanoclay materials, and, optionally, one or more contrast agents to make a composition provided herein. In some cases, a hydrogel composition provided herein can be made as described in Example 1.

Also provided herein are methods for using one or more hydrogel compositions provided herein (e.g., a hydrogel composition including decellularized ECM and one or more nanoclay materials). In some cases, one or more hydrogel compositions provided herein can be used for embolization of one or more blood vessels (e.g., permanent arterial embolization) within a mammal (e.g., a human). For example, one or more hydrogel compositions provided herein can be delivered to one or more blood vessels within a mammal for embolization of the blood vessel(s). In some cases, one or more hydrogel compositions provided herein can be used for embolization without fragmentation of the delivered hydrogel compositions. In some cases, one or more hydrogel compositions provided herein can be used for embolization without migration of the hydrogel compositions. In some cases, one or more hydrogel compositions provided herein can be used for embolization having a recanalization rate of less than about 35% (e.g., less than about 30%, less than about 25%, less than about 20%, less than about 15%, or less than about 10%).

In some cases, one or more hydrogel compositions provided herein can be used for enhanced vascular healing of one or more blood vessels within a mammal (e.g., a human). For example, one or more hydrogel compositions provided herein can be delivered to one or more blood vessels within a mammal for enhanced vascular healing of the blood vessel(s).

In some cases, one or more hydrogel compositions provided herein (e.g., a hydrogel composition including decellularized ECM and one or more nanoclay materials) can be delivered to one or more blood vessels within a mammal (e.g., a human) to reduce or eliminate blood flow within the blood vessel(s). For example, one or more hydrogel compositions provided herein can be delivered to one or more blood vessels within a mammal (e.g., a human) to reduce blood flow within the blood vessel(s) by for example, 10, 20, 30, 40, 50, 60, 70, 80, 90, 95, or more percent. For example, one or more hydrogel compositions provided herein can be delivered to one or more blood vessels within a mammal (e.g., a human) to eliminate blood flow within the blood vessel(s) (e.g., to reduce the blood flow to 0 cm/second.

In some cases, one or more hydrogel compositions provided herein (e.g., a hydrogel composition including decellularized ECM and one or more nanoclay materials) can be delivered to one or more blood vessels within a mammal (e.g., a human) to induce clotting within the blood vessel(s). For example, one or more hydrogel compositions provided herein can be delivered to one or more blood vessels within a mammal (e.g., a human) to induce clotting within the blood vessel(s) in less than about 10 minutes.

In some cases, one or more hydrogel compositions provided herein (e.g., a hydrogel composition including decellularized ECM and one or more nanoclay materials) can be delivered to one or more blood vessels within a mammal (e.g., a human) to increase collagen deposition at the delivery site. For example, one or more hydrogel compositions provided herein can be delivered to one or more blood vessels within a mammal (e.g., a human) to increase collagen deposition at the delivery site by, for example, 10, 20, 30, 40, 50, 60, 70, 80, 90, 95, or more percent.

In some cases, one or more hydrogel compositions provided herein (e.g., a hydrogel composition including decellularized ECM and one or more nanoclay materials) can be delivered to one or more blood vessels within a mammal (e.g., a human) to increase angiogenesis at the delivery site. For example, one or more hydrogel compositions provided herein can be delivered to one or more blood vessels within a mammal (e.g., a human) to increase angiogenesis at the delivery site by, for example, 10, 20, 30, 40, 50, 60, 70, 80, 90, 95, or more percent.

In some cases, one or more hydrogel compositions provided herein (e.g., a hydrogel composition including decellularized ECM and one or more nanoclay materials) can be delivered to one or more blood vessels within a mammal (e.g., a human) to increase cellular proliferation at the delivery site. For example, one or more hydrogel compositions provided herein can be delivered to one or more blood vessels within a mammal (e.g., a human) to increase cellular proliferation at the delivery site by, for example, 10, 20, 30, 40, 50, 60, 70, 80, 90, 95, or more percent.

In some cases, one or more hydrogel compositions provided herein (e.g., a hydrogel composition including decellularized ECM and one or more nanoclay materials) can be delivered to one or more blood vessels within a mammal (e.g., a human) having a bleeding disorder to treat the mammal. For example, a hydrogel composition provided herein can be delivered to one or more blood vessels feeding one or more tumors within the mammal to reduce or eliminate blood flow associated with the bleeding disorder. Examples of bleeding disorders that can be treated as described herein (e.g., by delivering a hydrogel composition including decellularized ECM and one or more nanoclay materials to one or more blood vessels within a mammal) include, without limitation, hemorrhage (e.g., non-traumatic hemorrhage and traumatic hemorrhage), saccular aneurysms, vascular malformations, and endoleak management.

In some cases, one or more hydrogel compositions provided herein (e.g., a hydrogel composition including decellularized ECM and one or more nanoclay materials) can be delivered to one or more blood vessels within a mammal (e.g., a human) having one or more tumors to treat the mammal. For example, a hydrogel composition provided herein can be delivered to one or more blood vessels feeding one or more tumors within the mammal to reduce or eliminate blood flow to the tumor(s). In some cases, a tumor can be a malignant tumor. In some cases, a tumor can be a benign tumor. Examples of tumors that can be treated as described herein (e.g., by delivering a hydrogel composition including decellularized ECM and one or more nanoclay materials to one or more blood vessels within a mammal) include, without limitation, hepatic tumors, uterine fibroids, and prostate tumors. For example, one or more hydrogel compositions provided herein can be delivered to one or more blood vessels feeding one or more tumors within a mammal (e.g., a human) to reduce the size (e.g., volume) of the tumor(s) by for example, 10, 20, 30, 40, 50, 60, 70, 80, 90, 95, or more percent.

In some cases, when one or more hydrogel compositions provided herein (e.g., a hydrogel composition including decellularized ECM and one or more nanoclay materials) are delivered to one or more blood vessels within a mammal (e.g., a human), the mammal can experience minimal or no complications associated with embolization. Examples of complications associated with embolization include, without limitation, vasospasm, thrombosis, dissections, rupture, stroke, infarction, and abscess.

One or more hydrogel compositions provided herein (e.g., a hydrogel composition including decellularized ECM and one or more nanoclay materials) can be delivered to one or more blood vessels within any type of mammal. In some cases, a mammal (e.g., a human) can be anticoagulated (e.g., can be taking one or more anticoagulants). In some cases, a mammal (e.g., a human) can be coagulopathic (e.g., can have a bleeding disorder in which the mammal’s blood’s ability to coagulate is impaired). Examples of mammals that can have one or more hydrogel compositions provided herein delivered to one or more blood vessels within the mammal include, without limitation, humans, non-human primates such as monkeys, dogs, cats, horses, cows, pigs, sheep, mice, rats, and rabbits.

One or more hydrogel compositions provided herein (e.g., a hydrogel composition including decellularized ECM and one or more nanoclay materials) can be delivered to any type of blood vessel within a mammal (e.g., a human). In some cases, a blood vessel can be a diseased blood vessel. In some cases, a blood vessel can be an injured blood vessel. Examples of types of blood vessels into which a hydrogel composition provided herein can be delivered include, without limitation, arteries, veins, and capillaries. When one or more hydrogel compositions provided herein are delivered to an artery, the artery can be any artery within a mammal (e.g., a human) such as a renal artery or an iliac artery.

One or more hydrogel compositions provided herein (e.g., a hydrogel composition including decellularized ECM and one or more nanoclay materials) can be delivered to any size blood vessel within a mammal (e.g., a human). In some cases, a blood vessel can have a diameter (e.g., a luminal diameter) of from about 8 microns to about 25,000 microns (2.5 cm).

When delivering one or more hydrogel compositions provided herein (e.g., a hydrogel composition including decellularized ECM and one or more nanoclay materials) to one or more blood vessels within a mammal (e.g., a human), any appropriate method of delivery can be used. In some cases, one or more hydrogel compositions provided herein can be administered to one or more blood vessels within a mammal (e.g., a human) by injection directly to a blood vessel (e.g., a blood vessel in need of embolization). In some cases, one or more hydrogel compositions provided herein can be delivered to one or more blood vessels within a mammal (e.g., a human) by catheter-directed delivery (e.g., via a catheter inserted into a blood vessel in need of embolization). When one or more hydrogel compositions provided herein are delivered to one or more blood vessels within a mammal (e.g., a human) by catheter-directed delivery any type of catheter can be used (e.g., a Bernstein catheter, a microcatheter, a Cobra catheter, a Fogarty balloon, and a ProGreat catheter). When one or more hydrogel compositions provided herein are delivered to one or more blood vessels within a mammal (e.g., a human) by catheter-directed delivery any size catheter can be used. For example, one or more hydrogel compositions provided herein can be administered to one or more blood vessels within a mammal (e.g., a human) using a catheter having a size of from about 2.8 French to about 5 French.

One or more hydrogel compositions provided herein (e.g., a hydrogel composition including decellularized ECM and one or more nanoclay materials) can be administered to one or more blood vessels within a mammal (e.g., a human) at any delivery rate. For example, one or more hydrogel compositions provided herein can be delivered to one or more blood vessels within a mammal (e.g., a human) at a rate of from about 1 mL/minute to about 3 mL/minute.

Any amount of one or more hydrogel compositions provided herein (e.g., a hydrogel composition including decellularized ECM and one or more nanoclay materials) can be delivered to one or more blood vessels within a mammal (e.g., a human). For example, from about 1 cc to about 3 cc of one or more hydrogel compositions provided herein can be administered to one or more blood vessels within a mammal (e.g., a human).

In some cases, after one or more hydrogel compositions provided herein (e.g., a hydrogel composition including decellularized ECM and one or more nanoclay materials) are used for embolization of one or more blood vessels within a mammal (e.g., a human), the hydrogel composition(s) can be retrieved from the blood vessel(s). For example, after one or more hydrogel compositions provided herein are delivered to one or more blood vessels within a mammal for embolization of the blood vessel(s), the hydrogel composition can be retrieved to increase (e.g., restore) blood flow through the blood vessel(s). Any appropriate method can be used to retrieve one or more hydrogel compositions provided herein from one or move blood vessels within a mammal (e.g., a human). For example, aspiration catheters can be used to retrieve one or more hydrogel compositions provided herein from one or more blood vessels within a mammal (e.g., a human).

In some cases, one or more hydrogel compositions provided herein (e.g., a hydrogel composition including decellularized ECM and one or more nanoclay materials) can be delivered to one or more blood vessels within a mammal (e.g., a human) as the sole active agent used for embolization.

In some cases, one or more hydrogel compositions provided herein (e.g., a hydrogel composition including decellularized ECM and one or more nanoclay materials) can be delivered to one or more blood vessels within a mammal (e.g., a human) in combination with one or more additional agents used for embolization. For example, one or more hydrogel compositions provided herein can be delivered to one or more blood vessels within a mammal (e.g., a human) in combination with solid embolic materials (e.g., a coils, particles, foam, a plug, microspheres, and/or beads), liquid embolic materials (e.g., butyl cyanoacrylate (n-BCA), and Onyx®).

In cases where one or more hydrogel compositions provided herein (e.g., a hydrogel composition including decellularized ECM and one or more nanoclay materials) are used in combination with additional agents used for embolization, the one or more additional agents can be administered at the same time (e.g., in the same composition or in separate compositions) or independently. For example, one or more hydrogel compositions provided herein can be administered first, and the one or more additional agents administered second, or vice versa.

### EXAMPLES

### Example 1: Bioactive Tissue Derived Nanocomposite Hydrogel for Permanent Arterial Embolization and Enhanced Vascular Healing

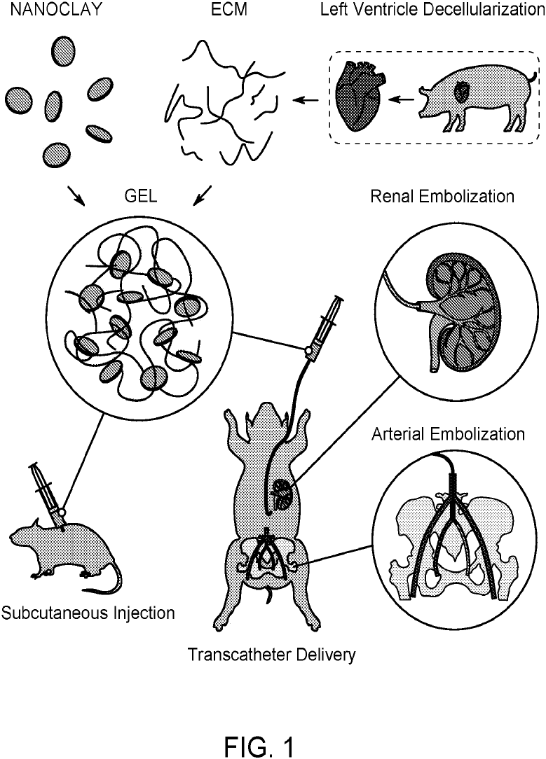

The Example describes the development of a unique class of ECM derived biohybrid nanocomposites to be used as multifunctional embolic agents. In order to overcome the limitations of currently used embolic agents, novel nanocomposite gels comprised of decellularized ECM from the left ventricle of the porcine heart and Laponite^{®} nanoclay (NC) were engineered. Synthetic NCs are highly charged nanodisks with positive charges along the rim and negative charges at the faces. This anisotropic charge distribution endows NC the ability to form self-assembled structures with antimicrobial properties (Gaharwar et al., ACS Nano., 8:9833 (2014); and Rawat et al., Appl. Biochem. Biotechnol., 174:936 (2014)). By combining NC with ECM, the mechanical strength and antibacterial characteristics of the hydrogel were significantly increased. The design of ECM-NC hybrid nanocomposites integrated the biochemical and biomechanical cues from ECM and the mechanical strength from synthetic NC to promote constructive remodeling in embolization as explored in a porcine model (FIG. 1). The bioactive ECM-NC hydrogel enabled wide tunability providing a platform technology for next-generation in vivo embolic agents to treat a broad range of vascular diseases.

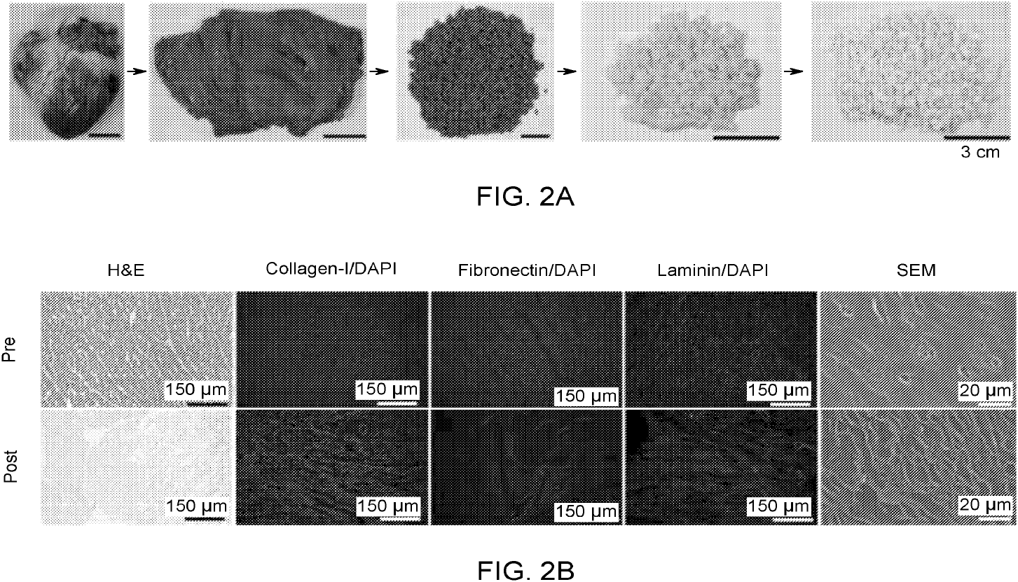

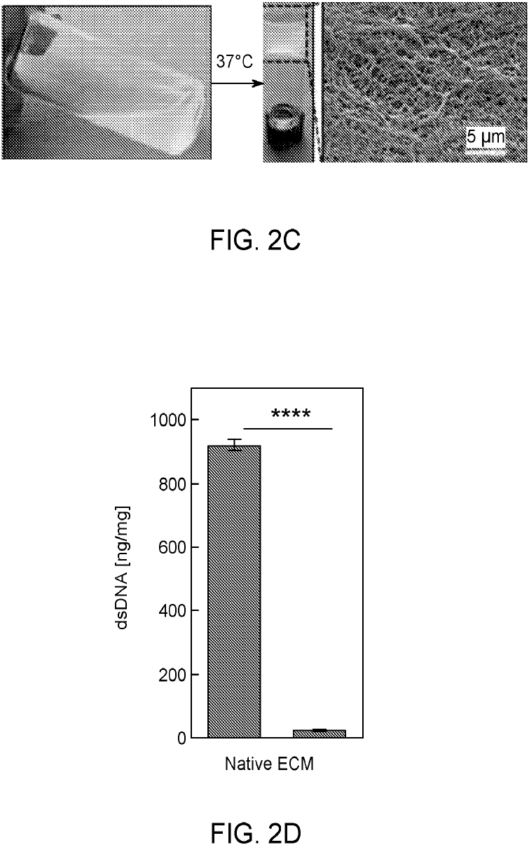

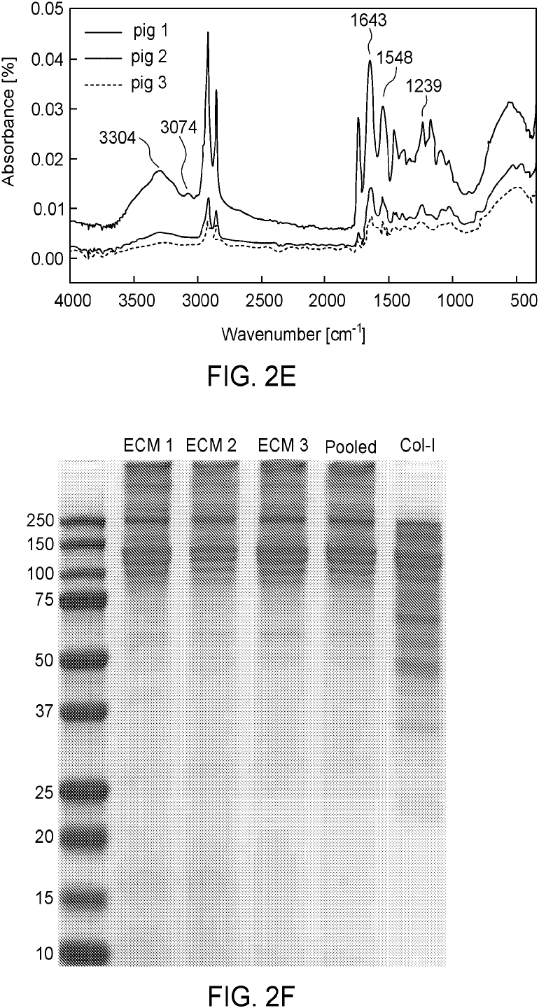

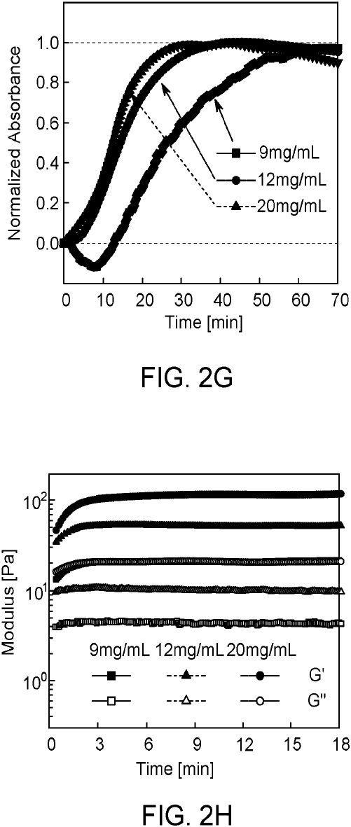

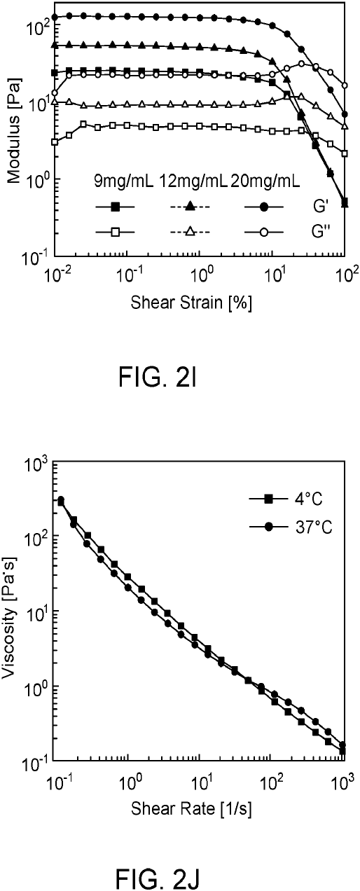

First, the cardiac ECM from the left ventricle of porcine hearts was produced and its microstructure and composition were characterized. To achieve this, the left ventricle was isolated, dissected into small pieces, decellularized and lyophilized (FIG. 2A). Decellularization was confirmed by staining for cells and nuclei with hematoxylin and eosin stain (H&E) and DAPI (FIG. 2B). Immunohistochemistry was also performed to confirm the preservation and structural integrity of collagen-I, fibronectin, and laminin after the decellularization process (FIG. 2B). These structural proteins have been shown to promote cell adhesion, migration, and proliferation for functional repair and tissue regeneration (Keane et al., Adv. Drug Del. Rev., 129:407 (2018)). Scanning electron microscopy (SEM) revealed the surface morphology of the decellularized cardiac tissue, showing its porous structure, compared to intact tissue (FIG. 2B). Next, the decellularized extracellular matrix was digested with pepsin. The solubilized matrix was neutralized with NaOH forming the ECM solution, which was subsequently mixed with NC to create the ECM-NC hydrogel. The ECM solution underwent a sol-gel transition at 37° C. and showed mesh-like microarchitecture (FIG. 2C). The DNA content of ECM was reduced by more than 98% (p<0.0001) compared to the native tissue and further confirmed that the decellularization process was complete, which is essential since cellular remnants such as DNA may provoke immunological reactions (FIG. 2D).

The chemical composition of ECM was assessed by Fourier-transform infrared spectroscopy (FTIR), while protein composition was analyzed by SDSpolyacrylamide gel electrophoresis (SDS-PAGE). FTIR spectra of ECMs obtained from three different pigs revealed consistent amide bands. SDS-PAGE of the same ECMs and pooled ECM samples demonstrated consistent purity and protein composition (FIG. 2F). Compared to rat-tail collagen-I, ECM showed bands at similar molecular weights; the protein composition of ECM mainly comprised of collagen proteins. These results suggested purity and homogeneity of the decellularized ECM isolated from the left ventricle of porcine heart, which was necessary for animal experiments.

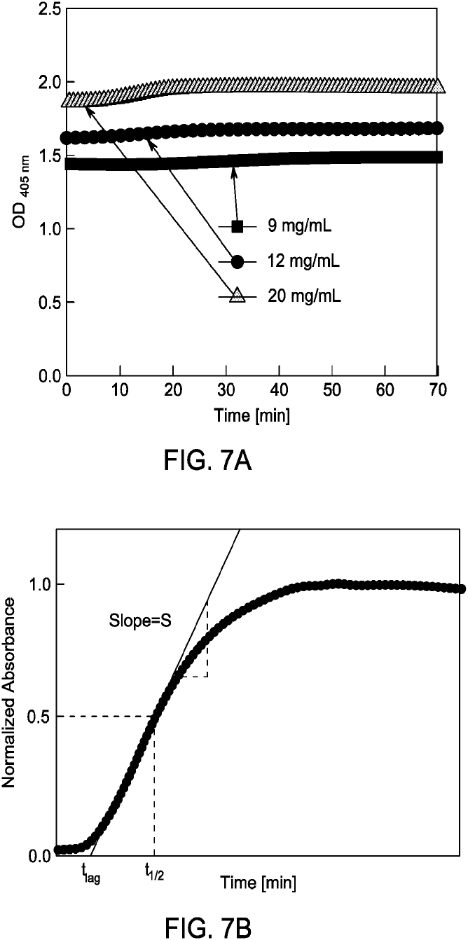

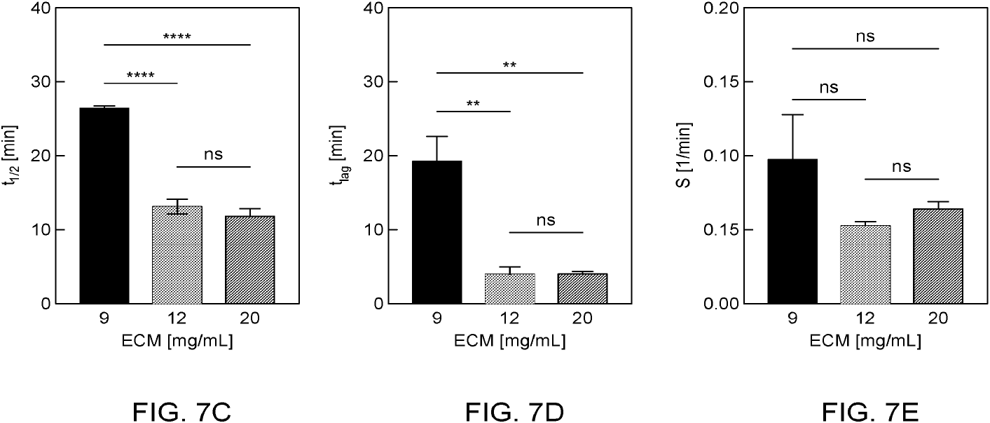

The gelation kinetics of the ECM solution was examined by both turbidimetry and rheology. Turbidimetric measurement was used to assess the changes of optical density in the ECM solution at 37° C., and the optical density was proportional to ECM concentration (FIG. 7A). The normalized gelation curve exhibited three distinct phases: a lag phase, an exponential growth phase, and a plateaued phase (FIG. 2G). The gelation data was used to calculate the time needed to reach 50% of the maximum turbidity absorbance (t_{½}), the rate of gelation (S), and the lagging time (t_{lag}) (duration before gelation started) (FIG. 7B). Similar gelation kinetics was observed between 12 mg/mL and 20 mg/mL ECM solution (FIGS. 7C, 7D, and 7E), whereas 9 mg/mLECM showed delayed gelation kinetics, possibly due to its lower concentration. In addition, time sweep measurement at 37° C. showed the G′ (storage modulus) of ECM was proportional to its concentration (FIG. 2H), which was further confirmed in oscillatory amplitude sweep (FIG. 21). The gelation process resulted from entropy driven self-assembly process of collagen fibrils, resulting in stronger crosslinking and network strengthening. Therefore, 20 mg/mL ECM solution was selected to make the ECM-NC gel due to its robust modulus enhancement upon increased temperature, as well as its high protein content and shear thinning properties (FIG. 2J) for effective embolization.

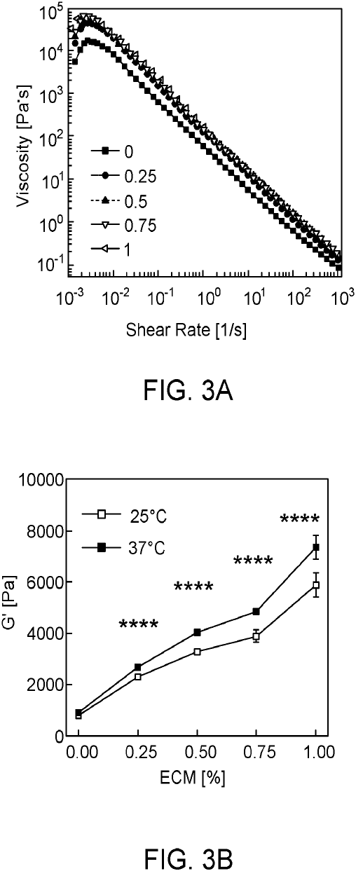

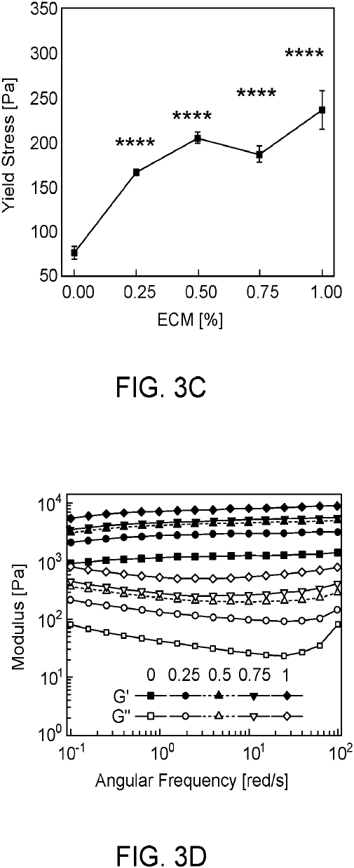

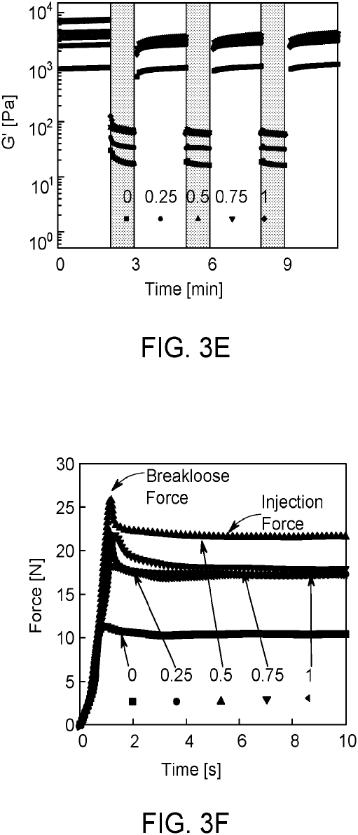

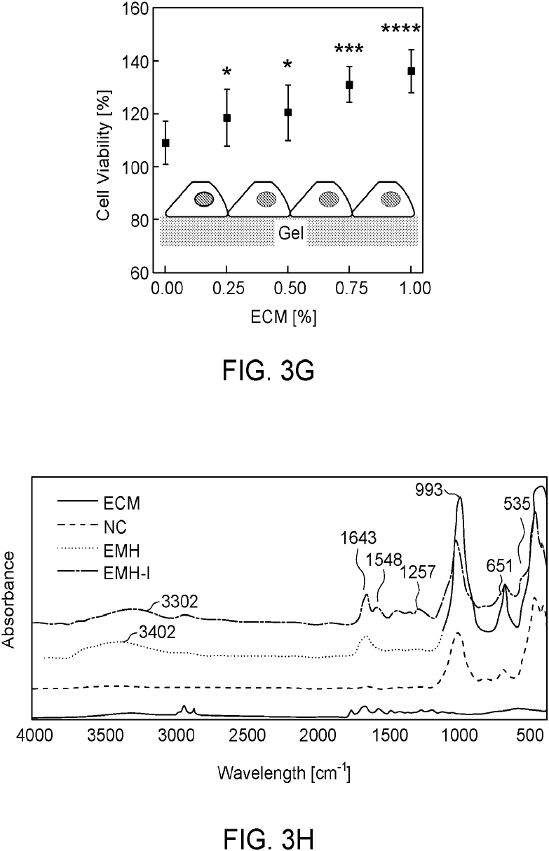

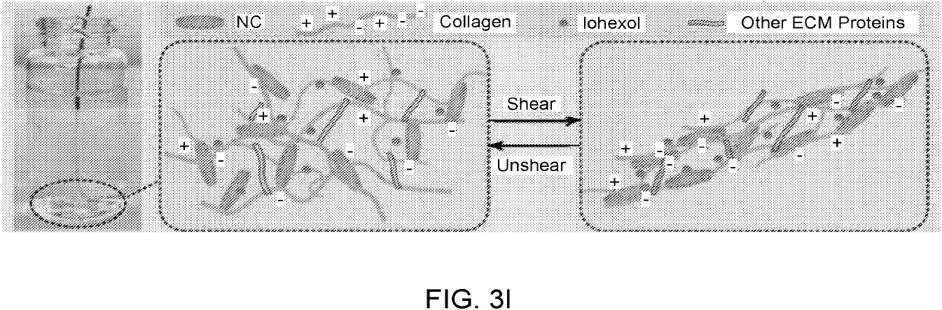

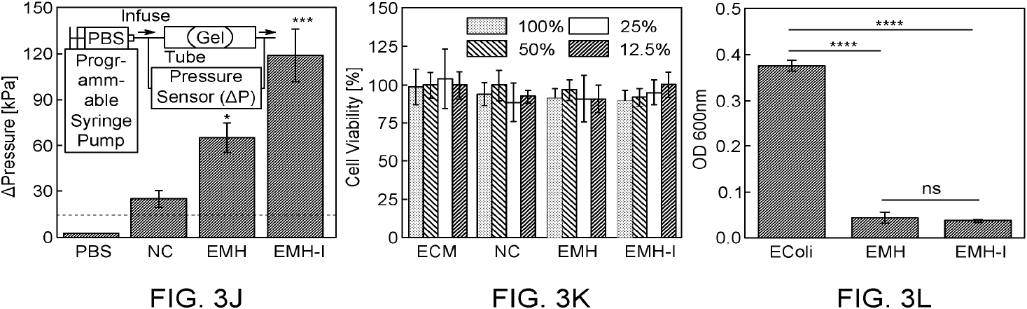

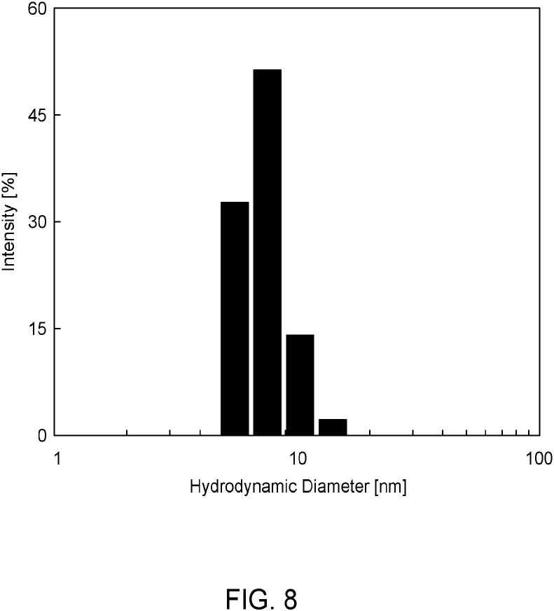

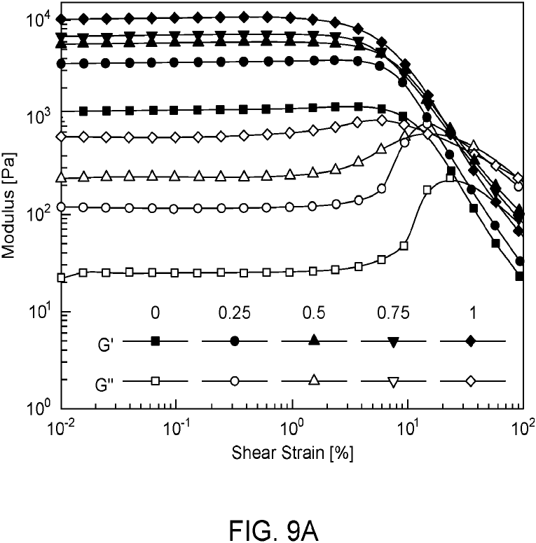

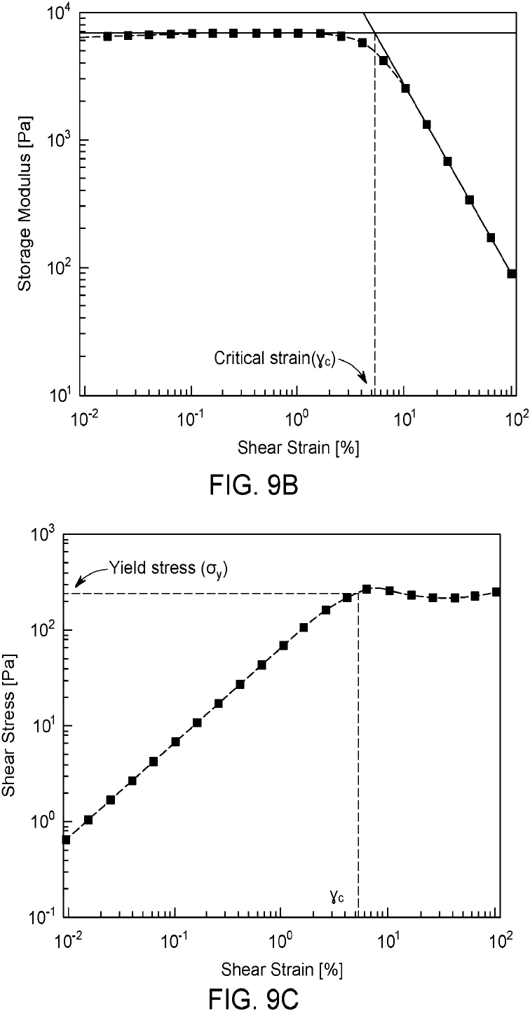

Next, ECM-NC nanocomposites were designed and fabricated by mixing 20 mg/mL ECM and 9 wt% Laponite® NC (with an average hydrodynamic size of 7 nm) at predetermined ratios (FIG. 8 and Table 1). The gels were labeled as xECMyNC, where x was ECM percentage, and y represented NC percentage. To investigate the role of ECM on mechanical and biological properties of xECMyNC, a group of xECM4.5NC composites were produced at constant NC content of 4.5 wt%, and a final ECM amount ranging from 0 wt% (0 mg/mL) to 1 wt% (12 mg/mL). Rheological measurements were performed to assess the viscoelastic properties of ECM-NC gels, which all exhibited shear-thinning behavior (FIG. 3A and Table 2). It indicated that the xECM4.5NC composites experienced decreased viscosity upon applied shear to facilitate transcatheter injection. The G′ of xECM4.5NC was analyzed to understand the interaction between ECM and NC. Oscillatory time sweep measurements indicated that G′ was proportional to the ECM content with constant NC amount (FIGS. 3B and 9A). Specifically, G′ of xECM4.5NC increased from 910 ± 4 Pa to 7327 ± 426 Pa (p<0.0001) at 37° C. with increasing ECM concentration from 0 to 1 wt%, suggesting enhanced mechanical strength and microstructure (FIG. 3B). Furthermore, G′ value of 1ECM4.5NC 37° C. was 25 % higher compared to its value at 25° C., whereas such enhancement was not evident in 0ECM4.5NC. The increase in G′ of 1ECM4.5NC may attribute to the ECM gelation at 37° C. since 1ECM4.5NC possessed a final ECM concentration of 12 mg/mL that demonstrated thermally trigged gelation (FIG. 2H). The yield stress also enhanced with the increasing amount of ECM from 76 ± 7 Pa (0ECM4.5NC) to 236 ± 22 Pa (1ECM4.5NC) (p<0.0001, FIG. 3C, FIGS. 9B and 9C), suggesting the gel’s enhanced ability to withstand shear stress along a vasculature wall and increased resistance to material breakdown. Angular frequency sweep measurements showed that all xECM4.5NC gels exhibited G′ values approximately 7-50 times higher than G″ (loss modulus) values, suggesting the formation of stable hydrogels (FIG. 3D). In addition, xECM4.5NC nanocomposites were recoverable, with excellent self-healing properties under oscillating strains, mimicking intermittent injection, and deployment process during embolization procedure (FIG. 3E). These results indicate that the ECM and NC could instantly form a stable network structure that is not disrupted under shear stress due to rapid, reversible electrostatic interactions.

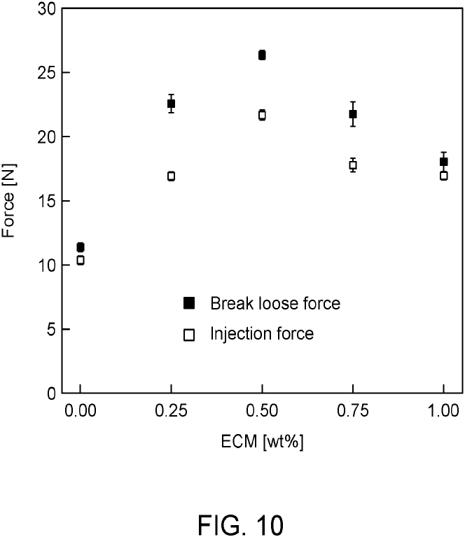

Although the rheological properties correlate with the material’s injectability, the injection force is the parameter that is directly related to physicians’ experience. Therefore, compression tests were performed to measure the injection force that was required to pass the gel (loaded in a syringe) through a clinically used 110 cm, 2.8 French (F) microcatheter. The force-time plot included the plunger-stopper break-loose force, representing the force the physician needed to overcome to initiate the plunger movement, and the injection force, representing the force required to sustain the plunger movement (FIG. 3F). All xECM4.5NC gels generated a break-loose force of 18-27 N, representing forces that can be easily injected by hand during catheter delivery (FIG. 3F and FIG. 10). These results are significant because they indicate the simplicity and the speed of embolic delivery, a critical desirable feature for physician adoption, and reducing costs. Today, to deliver coils, often special catheters, wires, and devices to release the coil are required; these additional products increase the cost of the procedure, and they prolong the procedure time increasing exposure to X-ray.

To investigate cell viability in contact with xECM4.5NC nanocomposites, L929 mouse fibroblast cells were seeded over the gels. The amount of viable cells increased proportionally to the amount of ECM. FIG. 3G demonstrates that 1ECM4.5NC (1 wt% ECM) exhibited the highest cell viability (136 ± 8%) (p<0.0001) compared to hydrogels containing less ECM. Since 1ECM4.5NC possessed the highest modulus, highest yield stress, and suitable injectability; this ECM hydrogel (EMH) was selected for further characterization.

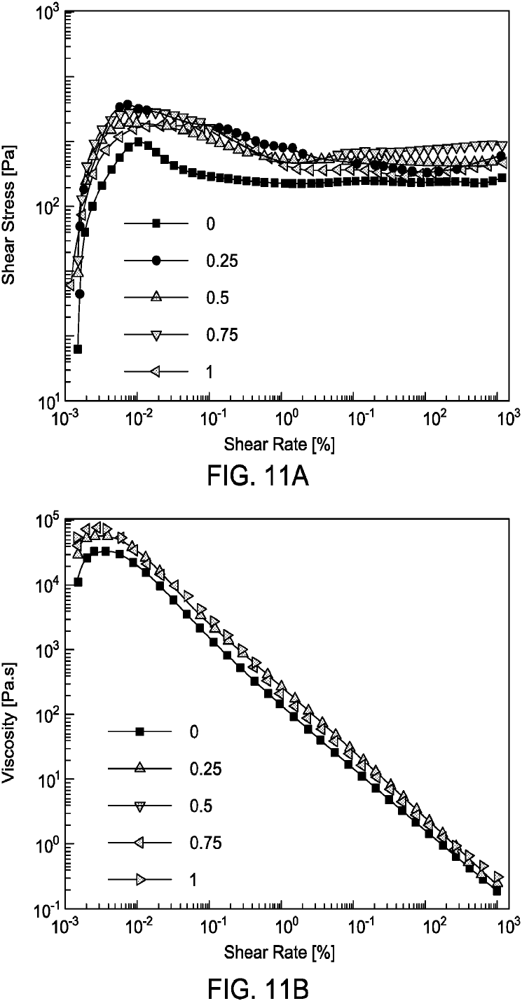

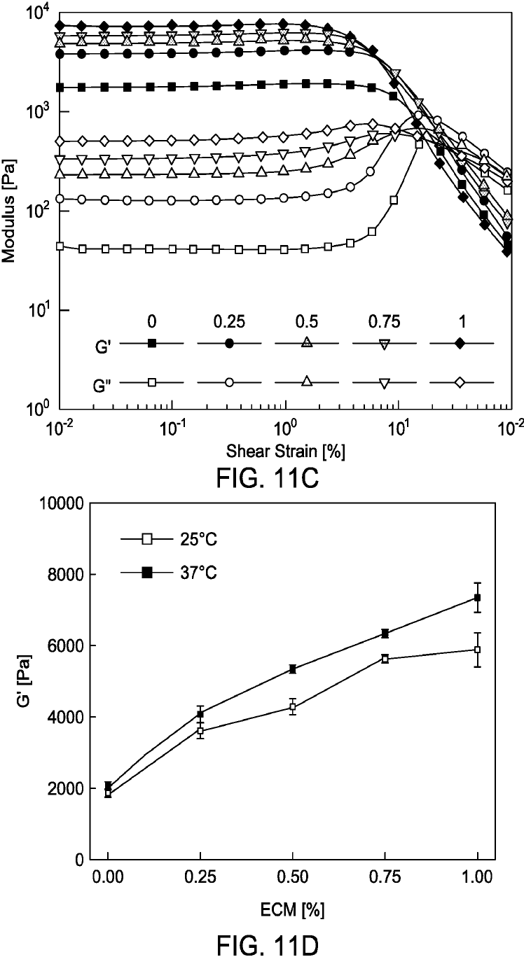

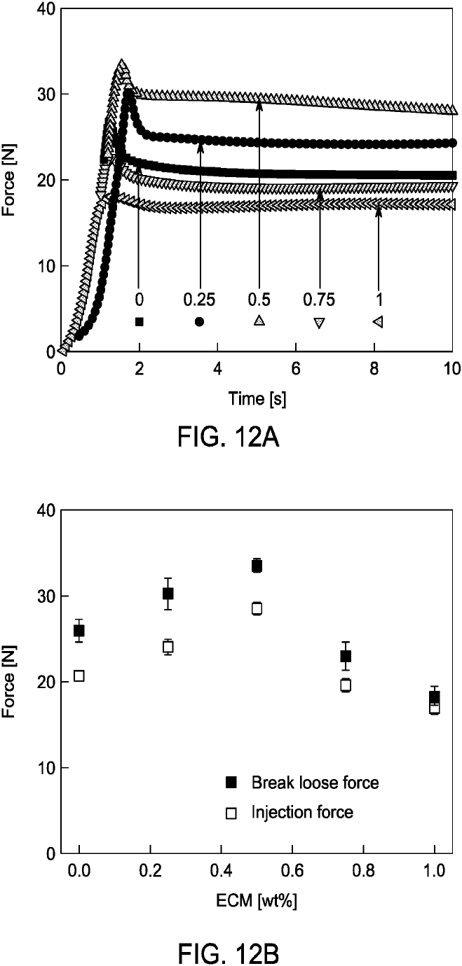

To better understand the degree of physical crosslinking on gel properties, ECM-NC nanocomposites were prepared with varied ECM/NC ratios, while keeping the total solid amount (NC and ECM) constant at 5.5 wt% (Table 3). In addition to being shear-thinning (FIGS. 11A and 11B), it is worth noting that G′ increased with increased ECM content and decreased NC amount (FIGS. 11C and 11D). Over threefold increase in G′ was observed between 0ECM5.5NC (1995 ± 84 Pa) and 1ECM4.5NC (7327 ± 426 Pa) (p<0.0001). These results suggested that the network’s strength results from the interaction between the NC and ECM, in addition to the solid content that determined the strength of a hydrogel in the context of 0ECM4.5NC. Nanocomposites fabricated with a constant solid concentration of 5.5 wt % showed excellent mechanical stability, recoverability, and injectability (FIGS. 11E, 11F, 12A, and 12B). These data demonstrated that by tuning the ratio between ECM and NC, a wide range of nanocomposites with varying mechanical properties for a variety of in vitro and in vivo applications was achieved.

Radiopacity is of great importance for any embolic agents for real-time tracking under X-ray based fluoroscopy; this allows accurate deployment preventing non-target embolization. Here, a clinically used aqueous contrast agent, iohexol (350 mgI/mL), was incorporated into the xECM4.5NC to form a radiopaque hydrogel, xECM4.5NC-I, with a final iohexol concentration of 27 wt% (Table 3). FTIR was used to confirm that ECM and iohexol were incorporated into the nanocomposite network (FIG. 3H). The characteristic peaks of NC were observed in the final composites (1ECM4.5NC and 1ECM4.5NC-I). A shoulder at 535 cm⁻¹ appeared in 1ECM4.5NC-I, which may be due to the aromatic ring C-H out-of-plane bending from iohexol, and 1257 cm⁻¹ corresponds to C-N stretch in aromatic amine group in iohexol. Negligible changes in peak positions along with similar FTIR patterns further revealed the noncovalent interactions between ECM, NC, and iohexol, suggesting the preservation of ECM protein complexes.

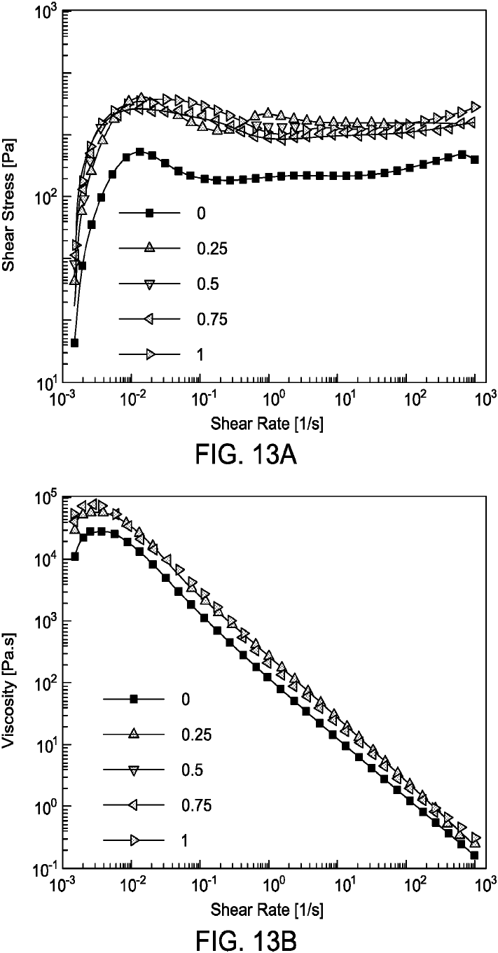

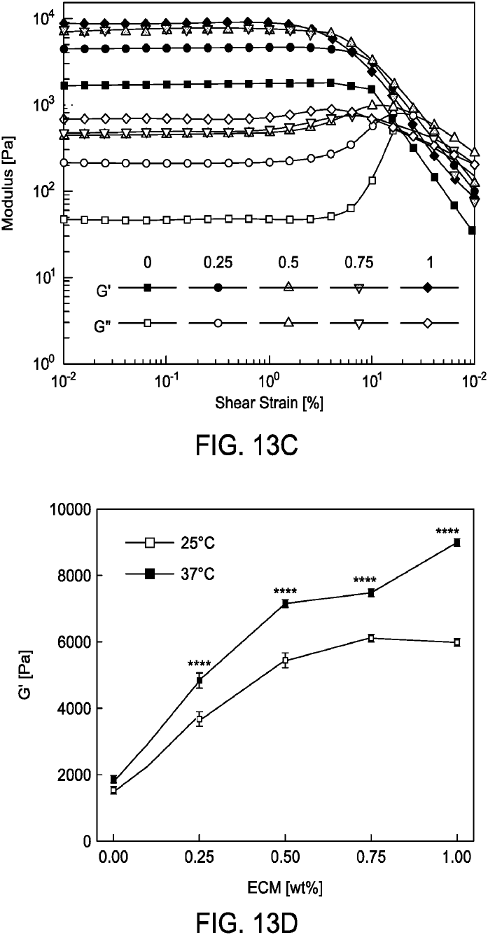

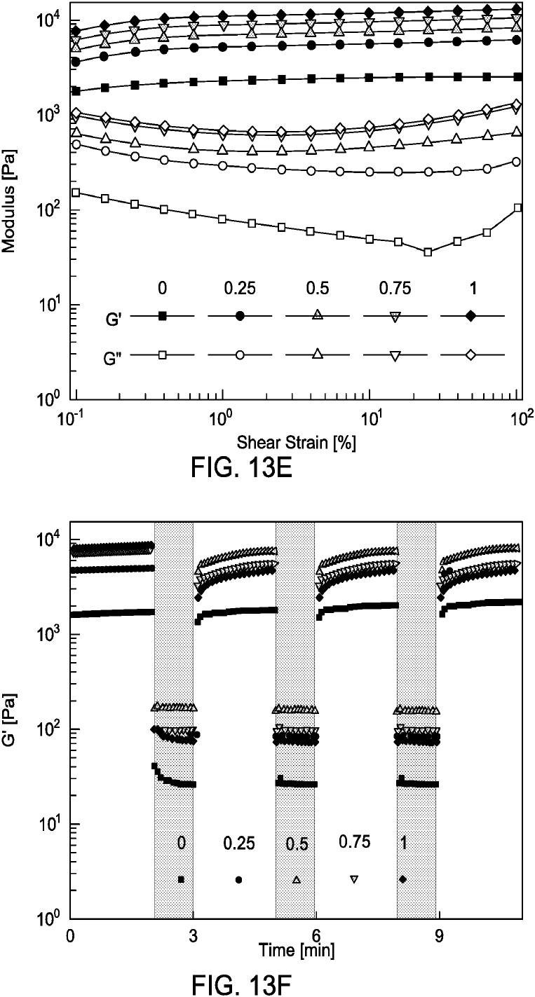

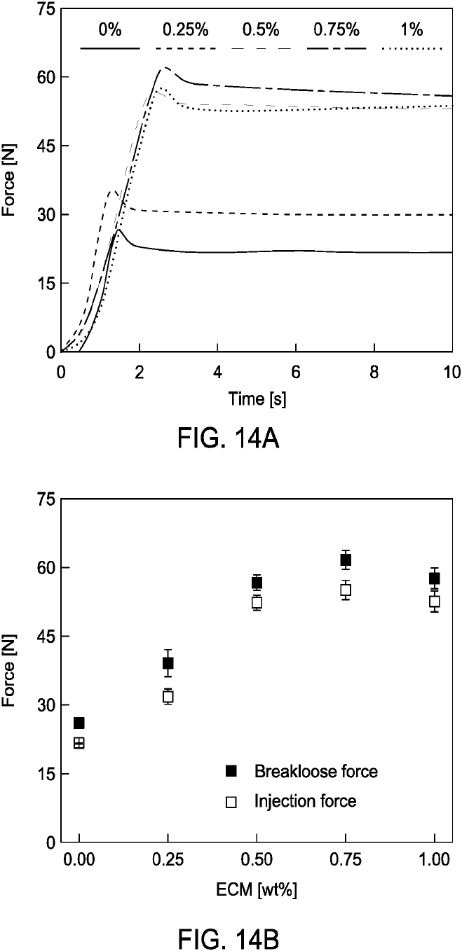

The effect of iohexol on the rheological property and bioactivity of nanocomposite hydrogels was further investigated. xECM4.5NC-I exhibited similar characteristics in mechanical properties compared to their radiolucent counterparts (FIG. 13). Specifically, the addition of contrast agent did not compromise the hydrogel’s shear-thinning properties, embolic strength, or recoverability (FIG. 13). Radiopaque nanocomposites with G′ in the range of 1808 ± 176 Pa (0ECM4.5NC-I) to 8984 ± 73 Pa (1ECM4.5NC-I) (p<0.0001) were generated (FIG. 13D). Overall, G′ of radiopaque hydrogels was increased due to the addition of iohexol, which enhanced hydrogen bonding and ionic interactions with the gel matrix, thereby strengthening the overall nanocomposite structure. In addition, iohexol may act as a physical obstacle to impede the movement of ECM protein chains and NC disks, further enhancing the network strength for higher G′ (FIG. 3I). The strengthening mechanism also resulted in higher injection forces (18 N-58 N) compared to their radiolucent counterparts, but still comfortably injectable by hand (FIG. 3I, FIGS. 14A, and 14B). It is interesting to note that for xECM4.5NC-I, an increased ECM concentration from 0.75 wt% to 1 wt% did not result in an increase in G′ at 25° C., but an enhancement of G′ at 37° C. (FIG. 12D). This could be due to the saturation of electrostatic interactions with the addition of iohexol. Therefore, when ECM content increased from 0.75 wt% to 1 wt%, the degree of crosslinking did not increase at 25° C., whereas self-assembly of collagen at 37° C. resulted in additional crosslinking that led to increased G′ of 1ECM4.5NC-I (FIG. 13D), suggesting its enhanced mechanical stability at physiological temperatures to support in vivo utilization. Therefore, radiopaque 1ECM4.5NC-I, namely EMH-I, was selected for further study.

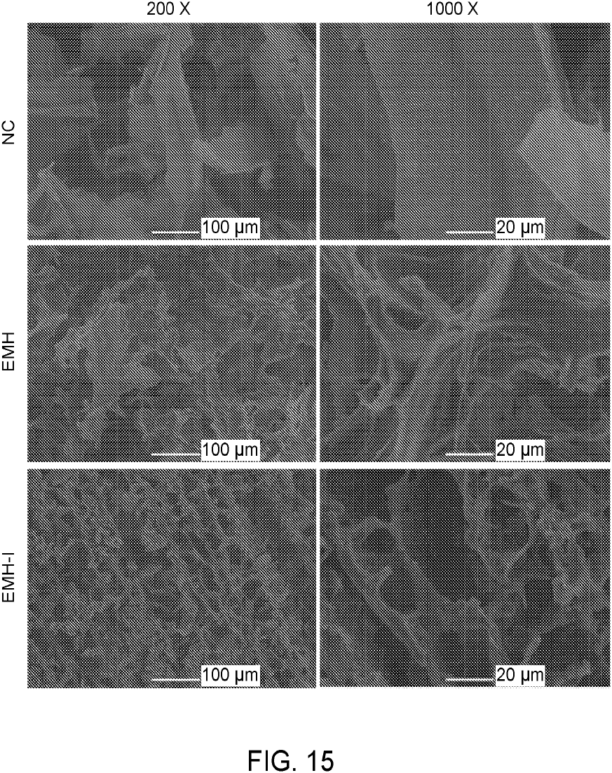

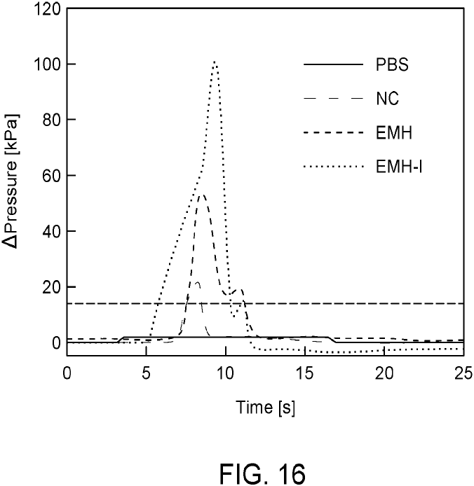

To understand the influence of ECM and iohexol on the hierarchical structure of NC, SEM was used to investigate the microarchitecture of NC, EMH, and EMH-I (FIG. 15). A marked structural reinforcement was observed when ECM was integrated into NC. The matrix structure was further strengthened and became more organized with the addition of iohexol. Since both EMH and EMH-I had pH values around 8, collagen-I molecules, the major components of the ECM matrix, were predicted to have both positive and negative charges along the chain, thereby interacting with both edge and face of NC disks (FIG. 3I). Compared to protein molecules (e.g., type A gelatin) carrying only positive charges that can interact purely with the faces of NC disks, the entangled conformation between ECM and NC offered a more compact and higher degree of hierarchical structures, therefore exhibiting significantly higher G′ compared to its gelatin counterpart. The mechanical strength of EMH and EMH-I was further confirmed with pressure displacement tests, which predicted the ability of EMH (65 ± 10 kPa) and EMH-I (119 ± 17 kPa) to withstand pressures much higher than physiological pressures (120 mmHg, equivalent to 16 kPa) (FIGS. 3J and 16). Furthermore, EMH-I demonstrated the feasibility to be retrieved in vitro. The retrievability endows EMH-I an important safety feature for rescuing non-target embolization and, for the first time, enabling temporary embolization. For example, the embolic agent could be removed from the internal iliac artery after treatment of pelvic hemorrhage avoiding buttock claudication.

In vitro tests revealed that both EMH and EMH-I were not cytotoxic. WST-1 assay was used as a qualitative evaluation of cytotoxicity by culturing L929 cells in hydrogel extracts for 24 hours at 37° C., showing no toxicity of the gels (FIG. 3K). Furthermore, FIG. 3L shows that the optical density of E. coli bacteria suspension incubated with EMH and EMH-I was reduced 88.5 ± 3.6 % (p<0.0001) and 90.4 ± 0.7 % (p<0.0001), respectively, suggesting the significant antibacterial effect of both materials. The antibacterial properties of EMH and EMH-I suggest that embolization could also be performed in patients with bacteremia or sepsis.

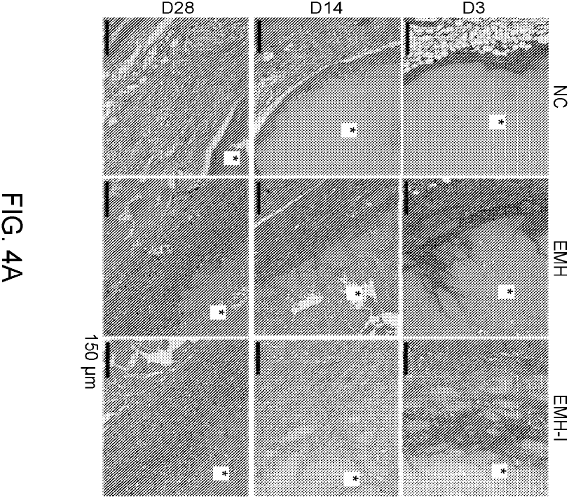

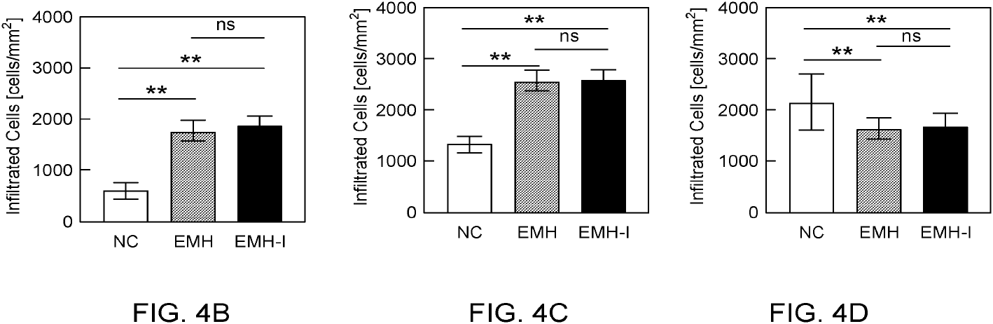

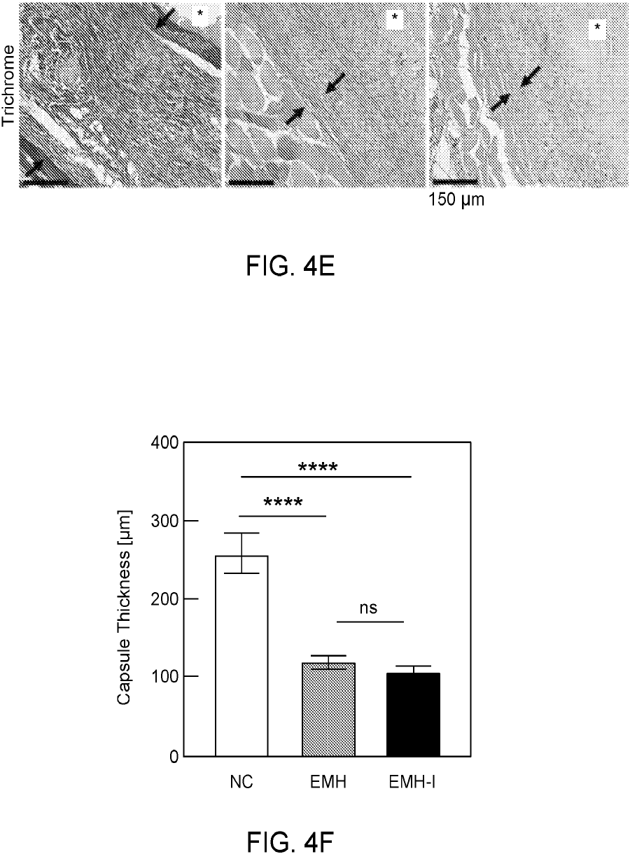

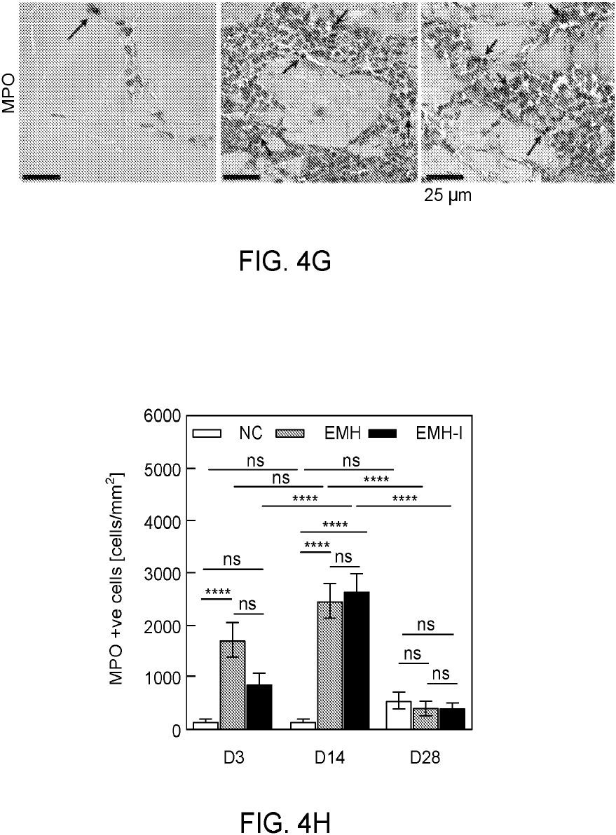

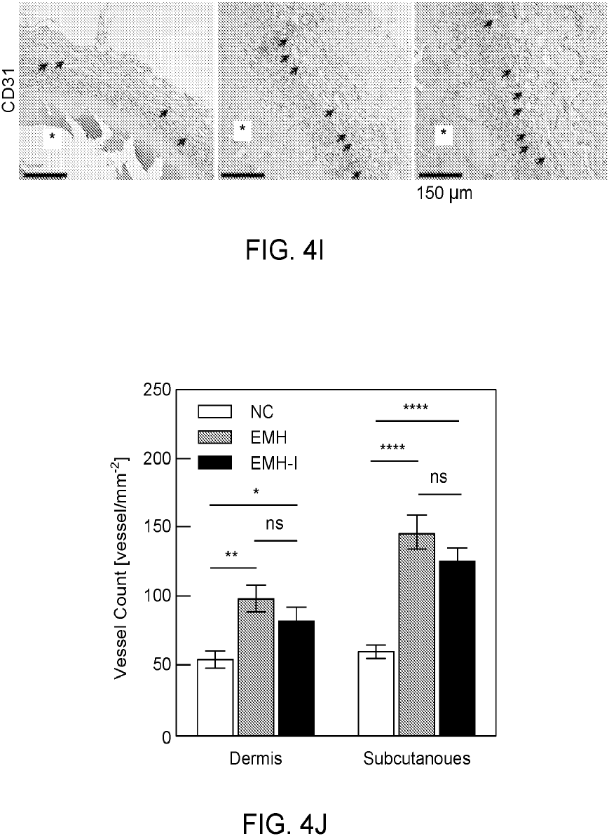

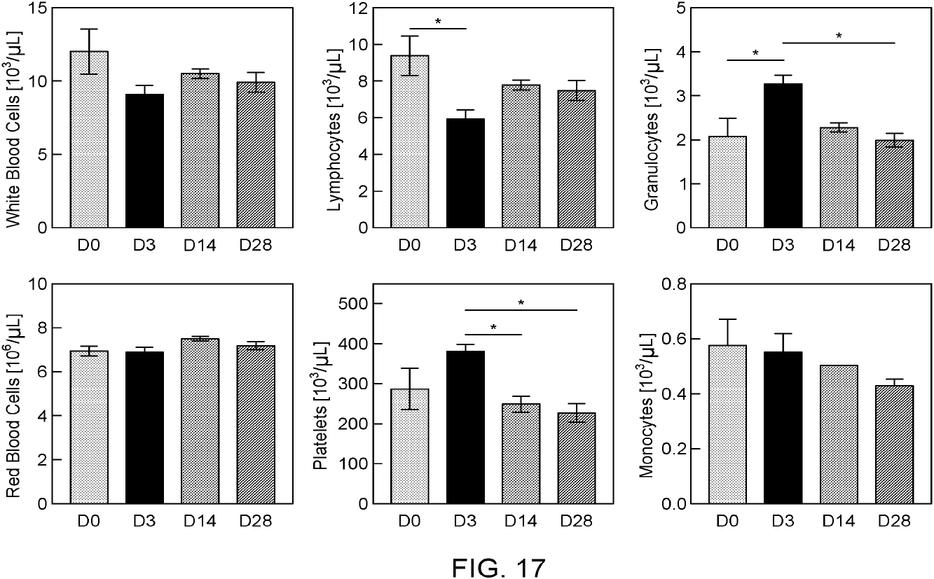

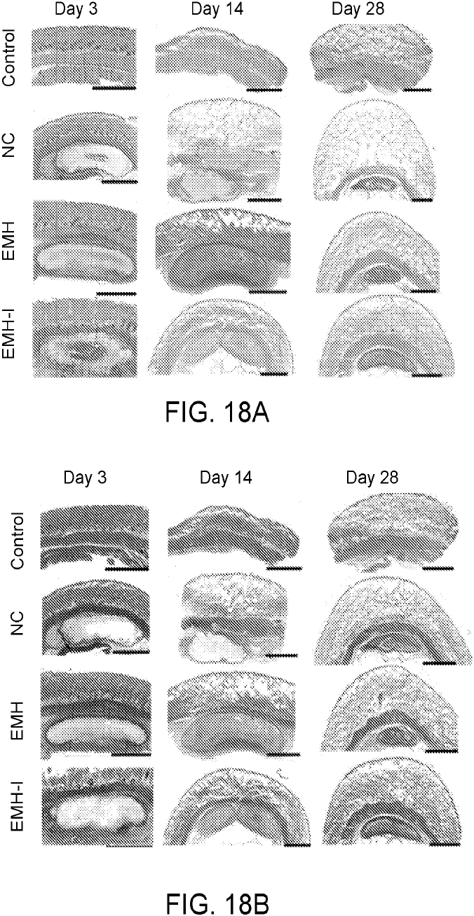

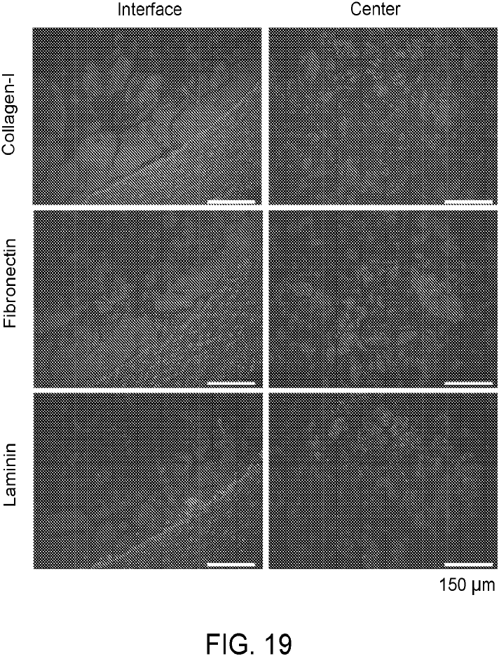

To investigate the host response in vivo, NC (control), EMH, or EMH-I were injected into the dorsal subcutaneous tissue of Sprague-Dawley rats; the implants were excised 0, 3, 14, and 28 days after injection. Complete blood counts (CBC) at 0, 3, 14, and 28 days (Table 4 and FIG. 17) across all groups were within the normal range. All implants decreased approximately 50% in cross-sectional area at D28 compared to D3 on histology (NC 15 ± 2 µm² at D3 and 8 ± 1 µm² at D28, p=0.07; EMH 16 ± 4 µm² at D3 and 7 ± 1 µm² at D28, p=0.01; EMH-I15 ± 4 µm² and D3 to 8 ± 2 µm² at D28, p=0.08) suggesting that all three materials degraded over time in vivo (FIGS. 18 and 19). Histological analysis revealed that cellular infiltration into NC was significantly lower at day 3 and 14 compared to EMH and EMH-I (p<0.01) (FIGS. 4A, 4B and 4C). However, the total number of infiltrating cells was similar for these three gels at day 28 (p=0.6 between NC and EMH, p=0.6 between NC and EMH-I, p=0.9 between EMH and EMH-I) (FIGS. 4A and 4D). Analysis of Masson’s trichrome staining for the thickness of the fibrotic capsule, defined as the dense collagen layer encapsulating the implant, is a standard measure of chronic inflammation to foreign materials following subcutaneous implantation (FIG. 4E). The formation of a fibrotic capsule in response to foreign material is associated with upregulated fibroblast proliferation and activation, which leads to excess collagen deposition at the tissue-material interface. On analysis of the capsular thickness on histology indicated that the measured thickness of the capsule in EMH (116 ± 8 µm) and EMH-I (102 ± 8 µm) explants were decreased by 55 % (p<0.0001) and 60 % (p<0.0001), respectively, compared to NC (257 ± 26 µm) (FIG. 4F). These results demonstrate that ECM can modulate chronic inflammatory events, and lead to faster host resolution of the tissue reaction and thinner capsule formation, which may be due to the downregulation of macrophage adhesion and activation.

Immunohistochemistry was performed to assess inflammation and angiogenesis using antibodies against myeloperoxidase (MPO, marker of neutrophil granulocytes) and CD31 (endothelial cell marker) respectively. MPO positive cells were remarkably higher at early stage (D3) in EMH (1722 ± 33 mm⁻²) (p<0.0001) and EMH-I (882 ± 197 mm⁻²) (p=0.3) explants, compared to NC (146 ± 33 mm⁻²) (FIG. 4G). At D14, there was a marked increase in newly recruited inflammatory cells in EMH and EMH-I compared to D3 (FIG. 4H). The physical cross-links in EMH and EMH-I can potentially be broken and displaced by migrating cells, resulting in enhanced cellular infiltration. By D28, inflammatory response subsided in EMH and EMH-I, indicating their biocompatibility (FIG. 4H). Furthermore, CD31 quantification of the blood vessel density at the hydrogel-tissue interface revealed that EMH (p=0.001 for dermis and p<0.0001 for subcutaneous site compared to NC) and EMH-I (p=0.04 for dermis and p<0.0001 for subcutaneous site compared to NC) demonstrated a positive effect on angiogenesis in the long-term (D28) on both dermis (100 ± 40 and 83 ± 35 vessels mm⁻² for EMH and EMH-I, respectively) and subcutaneous sites (147 ± 49 and 126 ± 38 vessels mm⁻² for EMH and EMH-I, respectively), compared to NC (55 ± 23 and 60 ± 18 vessels mm⁻² for dermis and subcutaneous sites). This enhanced angiogenesis suggested pro-regenerative properties of EMH and EMH-I, and confirmed that iohexol did not compromise biocompatibility or bioactivity of the material (p>0.05, FIG. 4J). Therefore, EMH-I was used in the subsequent large-animal studies since the presence of iohexol will allow its visibility under X-ray.

To demonstrate EMH-I’s feasibility and applicability for potential clinical use, EMH-I was delivered through standard clinical catheters to explore its efficacy in arterial embolization in a porcine model. Whether EMH-I can achieve instant embolization, remain at the site of release without migration or fragmentation avoiding non-target embolization, and whether it can enhance fibrosis of the arterial lumen to ensure that the occlusion is permanent was investigated. In addition, the performance of EMH-I embolization in anticoagulated animals was explored. Pigs in the non-survival group received 10,000 units of heparin intravenously (IV) and the pigs in the survival group received daily anti-platelet therapy. The goal was to embolize an immediate branch of the aorta, i.e., a first-order artery; these arteries are larger in diameter, more challenging to achieve complete occlusion using coils today, have higher flow rates and higher pressures. Clinical scenarios were chosen that would be challenging to occlude with the embolization tools available today in order to demonstrate the superior performance of EMH-I.

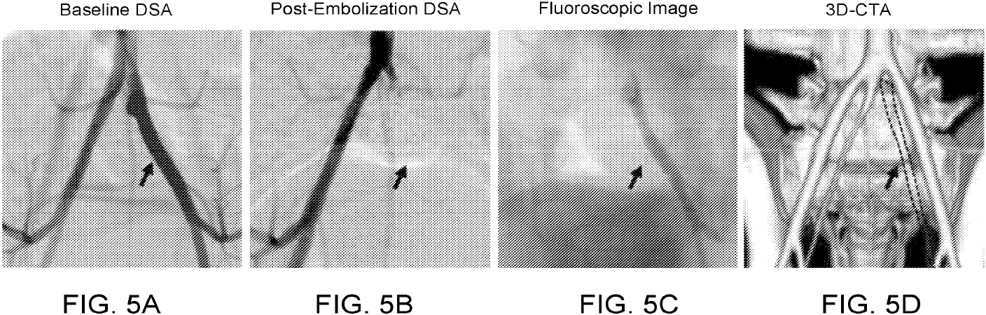

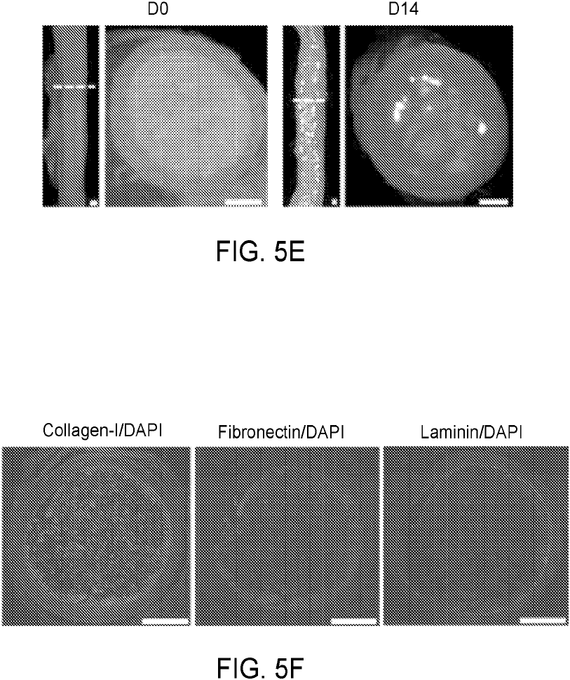

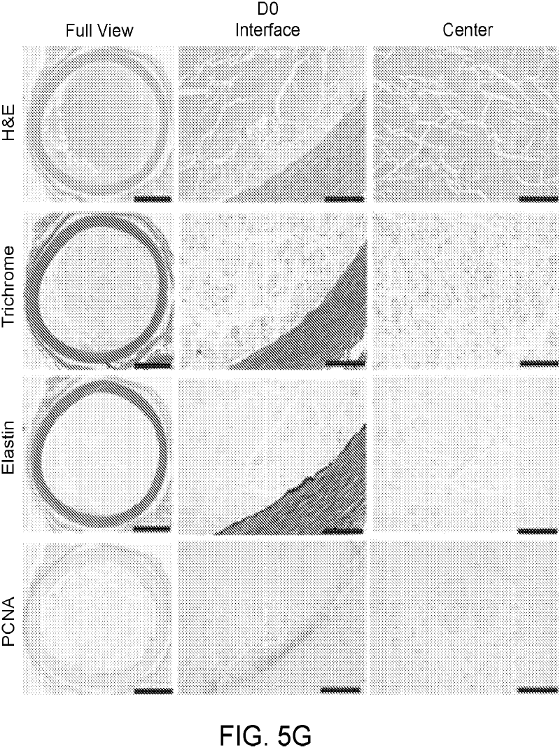

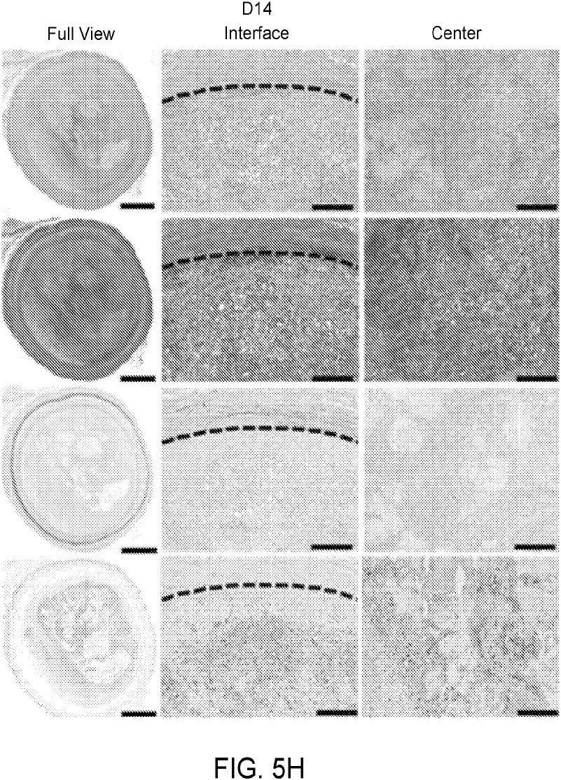

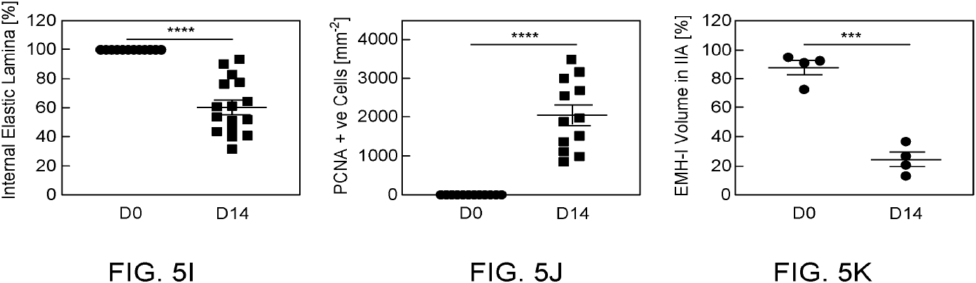

From a carotid artery access, a 5 French catheter was delivered to the distal aorta, and contrast- enhanced digital subtraction angiography (DSA) was performed showing the iliac arteries (FIG. 5A). Using a glidewire, the internal iliac artery (IIA) was catheterized with the 5 French catheter and embolization was performed (n=4); 1 mL syringe filled with EMH-I was connected to the catheter and, during real-time X-ray fluoroscopy, approximately a total of 3 mL of EMH-I was injected over 5-7 seconds creating an impenetrable cast of the artery. Subsequent DSA from the distal aorta immediately demonstrated the absence of flow in the IIA, showing complete occlusion (FIGS. 5B and 5C) despite having received IV heparin to achieve activated clotting time (ACT) values >300 s.

A subset of the animals was allowed to survive 14 days (n=4); CT angiography (CTA) just prior to necropsy demonstrated persistent occlusion of the IIA without any evidence for non-target embolization (FIG. 5D). Flow to the hindlimb and flow distal to the embolized IIA were preserved from cross-pelvic collaterals; in addition, CT evaluation of the whole body by a board-certified radiologist revealed unremarkable findings with no evidence for lymphadenopathy or any other pathology. C onsistent IIA embolization was achieved in all animals (FIGS. 21 and 22). Following necropsy, the IIA was harvested and further evaluated by high-resolution micro-computerized tomography (microCT) revealing material and tissue remodeling at D14. At D0, EMH-Iuniformly occluded the artery with homogenous enhancement of the EMH in the arterial lumen (FIG. 5E). At D14, the microCT enhancement pattern in the IIA lumen was heterogeneous (FIG. 5E), suggesting EMH-I degradation and artery remodeling.

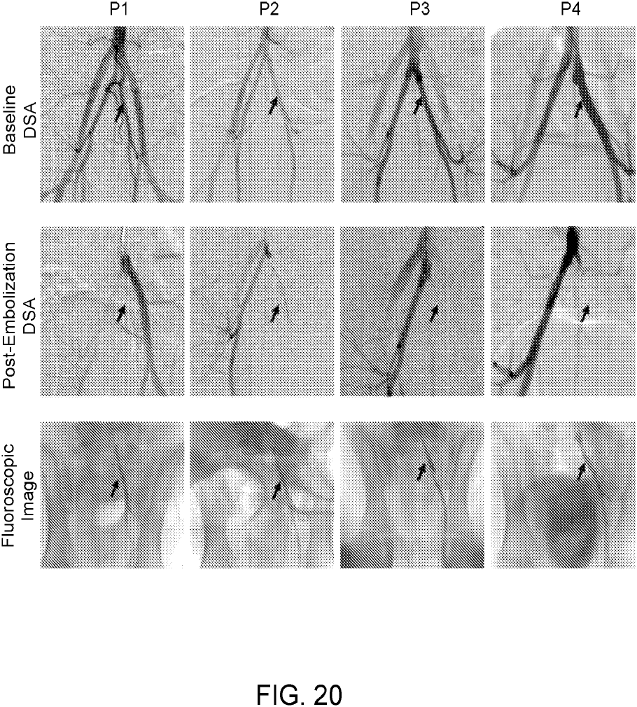

Following microCT imaging, the tissues were analyzed by histology and immunohistochemistry. Immunohistological staining of collagen-I, fibronectin, and laminin of the iliac artery at D0 confirmed the preservation of major ECM proteins in EMH-I in the embolized artery (FIGS. 5F and 20). Histologic evaluation of arteries treated with EMH-I was performed by a board-certified pathologist. At D0, the ECM appeared as a pale pink amorphous material expanding and occluding the arterial lumen (FIG. 5G). At D14, the arterial lumen remained entirely occluded, although the volume of ECM is reduced and partially replaced by a fibro-inflammatory process (FIG. 5H). This process included infiltration of the arterial lumen by myofibroblasts, macrophages, and neutrophils, with early collagen deposition. Evaluation of the arterial wall by trichrome and elastic stains demonstrated preserved arterial wall thickness.

Elastic stain showed disruption of elastic fibers at D14 (60 ± 5 %, p<0.0001) in the intima and media, but no significant injury to the smooth muscle layer was noted on trichrome stained slides (FIGS. 5G, 5H, and 5I). A significant amount of proliferating cell nuclear antigen (PCNA) positive cells were also observed (2048 ± 262 mm-², p<0.0001), indicating that the bioactive EMH-Ifacilitated cell proliferation in the surrounding microenvironment (FIG. 5J). Therefore, the histology evaluation performed 14 days following injection demonstrated that the arterial lumen remains completely occluded without evidence of recanalization. Morphologically, the hypercellular fibroinflammatory response observed in EMH-I samples appeared more robust than the response typically seen in an organizing thrombus. Subsequently, to determine the degradation rate of EMH-I within the arterial lumen, the volume of EMH-I was measured from reconstructed microCT images through segmentation by separating the material from connective tissue. By day 14, ECM-I in the arterial lumen was significantly reduced compared to day 0 samples (25 ± 5 % EMH-I remaining at D14; p=0.0001) (FIG. 5K) and was replaced by non-enhancing fibrotic tissue.

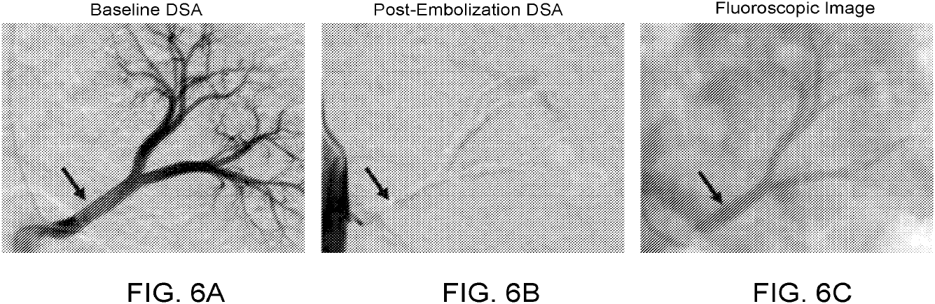

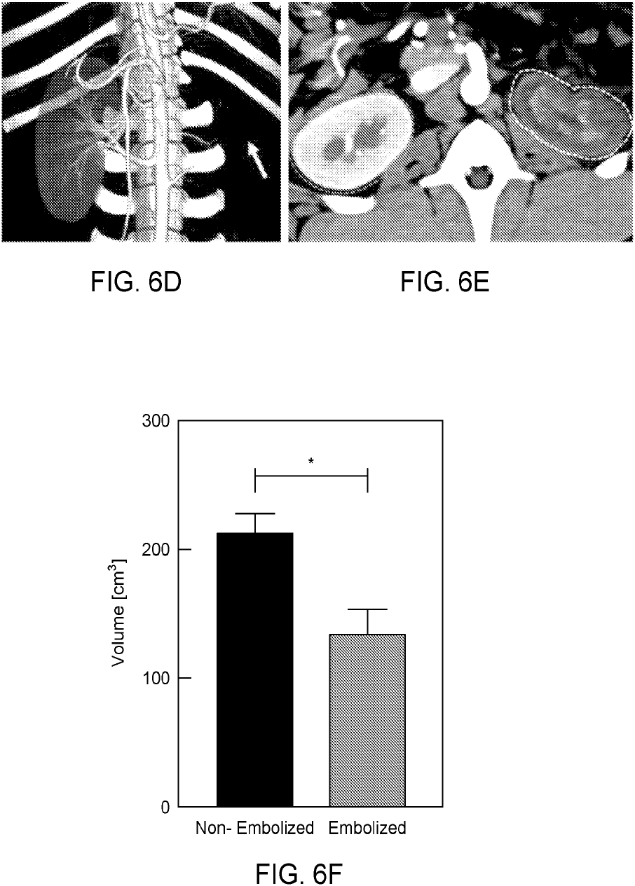

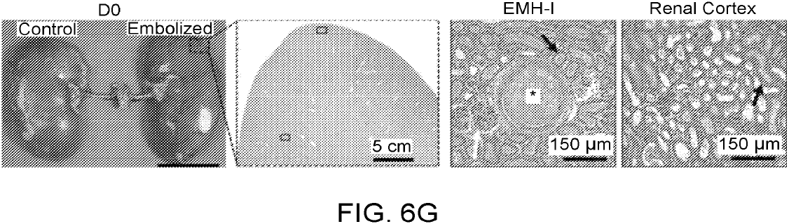

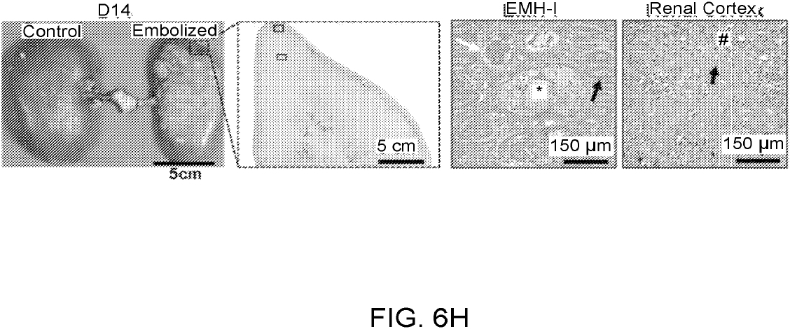

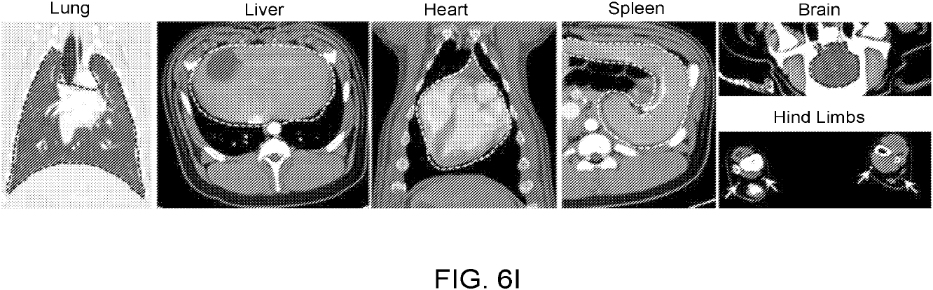

To investigate whether the embolic material leads to micro-fragmentation, an end-organ artery such as the main renal artery of the kidney was embolized. Any fragmentation from the embolized main renal artery would be detected by high-resolution microCT imaging and by histology. In addition, any recanalization of the main renal artery would show contrast enhancement of the renal parenchyma by CTA imaging. From a carotid artery access, a 5 French catheter was used to catheterize one of the main renal arteries, and DSA was performed demonstrating the normal renal arterial anatomy (FIGS. 6A and 23). From a distal renal artery position, approximately 2-3 mL of EMH-I was injected through the 5 French catheter causing immediate casting along the arterial lumen (FIG. 6B). Subsequent DSA images from the aorta demonstrated the absence of flow in the embolized renal artery with no contrast-enhancement of the kidney despite the animals receiving anticoagulation (FIG. 6C). A subset of these animals was allowed to survive 14 days (n=4). Prior to necropsy, contrast enhanced CTA was performed, demonstrating persistent occlusion of the embolized artery with no evidence for contrast enhancement of the renal parenchyma suggesting the absence of recanalization (FIGS. 6D, 6E, 23, and 24). A significant reduction (~36%) in kidney volume was also detectable post-embolization at D14 (213 ± 15 cm³ for non- embolized kidney, and 135 ± 20 cm³ for embolized kidney, p=0.02) (FIG. 6F). Following whole-body CT imaging, the embolized and the contralateral normal kidney were harvested (FIGS. 6G and 6H). These kidneys were further evaluated by microCT imaging, which revealed the absence of micro-emboli and complete occlusion of the renal artery (FIG. 25). On histology, there was no evidence of EMH-I in the cortex, suggesting that micro-emboli did not occur (FIGS. 6G and 6H). Histological analysis indicated the ability of EMH-I to penetrate vessel sizes down to 200 µm (FIG. 6H). The thinning of renal capsule, the destruction of tubules and glomeruli, and the fibrotic tissue in the parenchyma at D14 all indicated the absence of blood flow and the loss of physiological function of the kidney (FIGS. 6G and 6H).

In these minimally invasive embolization experiments, all pigs tolerated the embolization procedure without any signs of distress. Blood hematology and serum biochemistry results demonstrated the absence of any signs of infection and normal organ function, including renal and liver function at day 14 (Table 5). Vital organs, including lung, liver, spleen, heart, brain, and lower limb, as shown in whole-body CT scans (FIG. 26), were unremarkable. There was no evidence for lymphadenopathy, pulmonary emboli, or stroke, suggesting that EMH-I did not traverse the capillary bed of the embolized artery.

In conclusion, a novel class of bioactive, tissue-derived, mechanically robust, and radiopaque ECM-based nanocomposites for vascular embolization was developed. EMH-I has shear-thinning properties allowing it to be injected from a wide range of micro and standard clinical catheters for easy and rapid injection resulting in instant hemostasis. In comparison to embolic agents used today, EMH-I is a “one-size-fits-all” embolic agent that does not require additional wires, devices, or special catheters for use. EMH-I also has unique properties in that it is mechanically stable, achieving persistent occlusion without migration or fragmentation in first-order arteries. It is also antimicrobial and pro-regenerative. EMH-I achieved complete occlusion of the embolized arteries despite being anticoagulated; this is a desirable feature in an embolic agent as coils today fail because they rely on the bodies intrinsic ability to form a thrombus to occlude the coil mass inside the artery. These properties and its ease of use make the ECM-NC nanocomposite highly attractive for a wide range of embolization applications, such as treatment of aneurysms and vascular malformations. The novel hybrid design of integrating tissue-based biological functions from ECM proteins and mechanical strength from synthetic nanoclay represents a new direction in the endovascular treatment of vascular diseases.

TABLE 1

Summary of xECM4.5NC comprised of 4.5 wt % NC, and varying ECM amount from 0 wt % (3 mg/mL) to 1 wt % (12 mg/mL).

| Gel | ECM (wt%) | ECM (mg/mL) | NC (wt%) | ECM+NC (wt%) | Water (wt%) |
|---|---|---|---|---|---|
| 0ECM4.5NC | 0 | 0 | 4.5 | 4.5 | 95.5 |
| 0.25ECM4.5NC | 0.25 | 3 | 4.5 | 4.75 | 95.25 |
| 0.5ECM4.5NC | 0.5 | 6 | 4.5 | 5 | 95 |
| 0.75ECM4.5NC | 0.75 | 9 | 4.5 | 5.25 | 94.75 |
| 1ECM4.5NC | 1 | 12 | 4.5 | 5.5 | 94.5 |

TABLE 2

Composition summary of ECM-NC nanocomposite hydrogel comprised of a total amount of 5.5 wt% solid, with varying amount of ECM and NC.

| Gel | ECM (wt%) | NC (wt%) | ECM+NC (wt%) | Water (wt%) |
|---|---|---|---|---|
| 0ECM5.5NC | 0 | 5.5 | 5.5 | 94.5 |
| 0.25ECM5.25NC | 0.25 | 5.25 | 5.5 | 94.5 |
| 0.5ECM5NC | 0.5 | 5 | 5.5 | 94.5 |
| 0.75ECM4.75NC | 0.75 | 4.75 | 5.5 | 94.5 |
| 1ECM4.5NC | 1 | 4.5 | 5.5 | 94.5 |

TABLE 3

Summary of radiopaque xECM4.5NC-I gels comprised of 4.5 wt% NC, 27 wt% iohexol and varying ECM amount from 0 wt% (0 mg/mL) to 1 wt% (12 mg/mL).

| Gel | ECM (wt%) | ECM (mg/mL) | NC (wt%) | ECM+NC (wt%) | Iohexol (wt%) |
|---|---|---|---|---|---|
| 0ECM4.5NC-I | 0 | 0 | 4.5 | 4.5 | 27 |
| 0.25ECM4.5NC-I | 0.25 | 3 | 4.5 | 4.75 | 27 |
| 0.5ECM4.5NC-I | 0.5 | 6 | 4.5 | 5 | 27 |
| 0.75ECM4.5NC-I | 0.75 | 9 | 4.5 | 5.25 | 27 |
| 1ECM4.5NC-I | 1 | 12 | 4.5 | 5.5 | 27 |

TABLE 4

Summary of complete blood count (CBC) for subcutaneously injected rats. Rats were healthy, and no infection was observed. Each data point represents average ± standard error (n=4).

| Parameter | Day 0 | Day 3 | Day 14 | Day 28 |
|---|---|---|---|---|
| White Blood Cell (WBC) (10³/µL) | 12.1 ± 0.8 | 9.1 ± 0.3 | 10.6 ± 0.1 | 9.9 ± 0.3 |
| Lymphocyte (LYM) (10³/µL) | 9.4 ± 0.5 | 6.0 ± 0.3 | 7.8 ± 0.1 | 7.5 ± 0.3 |
| Monocyte (MONO) (10³/µL) | 0.6 ± 0.0 | 0.6 ± 0.0 | 0.5 ± 0.0 | 0.4 ± 0.0 |
| Granulocyte (GRAN) (10³/µL) | 2.1 ± 0.2 | 3.3 ± 0.1 | 2.3 ± 0.0 | 2.0 ± 0.1 |
| Red Blood Cell (RBC) (10⁶/µL) | 6.9 ± 0.1 | 6.9 ± 0.1 | 7.4 ± 0.0 | 7.1 ± 0.1 |
| Platelet (PLT) (10³/µL) | 284.3 ± 26.1 | 340.8 ± 19.0 | 246.2 ± 10.2 | 224.5 ± 11.9 |

TABLE 5

Complete blood count and biochemistry for pigs underwent embolization at D0 and D14. Pigs were healthy, and no infection was observed. ns, not significant; *p < 0.05, **p < 0.01, ***p < 0.005. Each data point represents the average ± standard error (n=4).

| Parameter | Day 0 | Day 14 | p value | Significance |
|---|---|---|---|---|
| Total Protein (TP) (g/dL) | 5.4 ± 0.2 | 7.0 ± 0.1 | 0.0005 | *** |
| Alkaline Phosphatase (ALP) (U/I) | 138.5 ± 23.7 | 126 ± 13.0 | 0.4165 | ns |
| Glucose (GLU) (mg/dL) | 116.0 ± 5.0 | 87.5 ± 3.7 | 0.0466 | * |
| Alanine Aminotransferase (ALT) (U/L) | 39.0 ± 5.8 | 43.0 ± 1.4 | 0.5697 | ns |
| Creatinine (CRE) (mg/dL) | 1.3 ± 0.2 | 1.6 ± 0.2 | 0.1514 | ns |
| Blood Urea Nitrogen (BUN) (mg/dL) | 12.5 ± 2.1 | 11.4 ± 1.1 | 0.5737 | ns |
| White Blood Cell (WBC) (10³/µL) | 13.4 ± 2.6 | 16.7 ± 5.8 | 0.4990 | ns |
| Lymphocyte (LYM) (10³/µL) | 7.5 ± 1.5 | 7.2 ± 1.4 | 0.7177 | ns |
| Monocyte (MONO) (10³/µL) | 0.7 ± 0.1 | 1.6 ± 0.8 | 0.3246 | ns |
| Granulocyte (GRAN) (10³/µL) | 5.2 ± 1.3 | 7.9 ± 4.1 | 0.5354 | ns |
| Hematocrit (HCT) (%) | 23.0 ± 1.9 | 22.7 ± 2.3 | 0.9423 | ns |
| Red Blood Cell (RBC) (10⁶/µL) | 4.9 ± 0.4 | 4.8 ± 0.5 | 0.9131 | ns |
| Platelet (PLT) (10³/µL) | 266.8 ± 36.2 | 292.3 ± 65.4 | 0.4662 | ns |

### EXPERIMENTAL INFORMATION

### Decellularization of Porcine Heart

Fresh porcine hearts were obtained from deceased pigs for decellularization. The left ventricle was collected and decellularized as described elsewhere (Johnson et al., Nanot., 22:494015 (2011)). Briefly, the cardiac tissue was first rinsed with DI water for 45 minutes, followed by 1 % (wt/vol) sodium dodecyl sulfate (SDS) (Fisher Scientific, Cat. # BP166) (in phosphate-buffered saline (PBS)), and washed for 4-5 days. SDS detergent was changed every 24 hours until the tissue was fully decellularized and turned completely white. Finally, decellularized cardiac tissue was washed in DI water for two days (with constant water change) to ensure the complete removal of SDS. A sample of cardiac tissue at day 0, 3, and 5 was collected and embedded in paraffin for histological analysis. The cardiac specimen was sectioned into 4 µm slices and stained with hematoxylin and eosin (H&E) to confirm the removal of cells. Lastly, the cardiac tissues were frozen at -80° C. before being lyophilized (Labconco, 0.120 mBar, and -50° C.) and stored at 4° C.

### Preparation of ECM

Lyophilized cardiac tissue was solubilized in 1 mg/mL pepsin (Sigma Aldrich, Cat. # 9001-75-6) (in 0.1 _{M} HCl), and underwent continuous digestion with vigorous agitation for 2 days to achieve a homogenous solution (25 mg/mL). The solution was then brought to pH 7.6 by adding 1 _{M} sodium hydroxide (NaOH), forming ECM solution. The final ECM solution (~20 mg/mL) was used freshly for characterization and ECM gel formation.

### ECM Protein Extraction

Protein in final ECM solution (after digestion and neutralization) was extracted into protein extraction buffer containing protease and phosphatase inhibitors. The samples were centrifuged at 12000 RPM at 4° C. for 10 minutes. The supernatant was transferred to a new tube for protein quantification using a Bicinchoninic Acid (BCA) Protein Quantification protein kit (Thermo Scientific, Prod. # 23225), according to the manufacturer’s instructions. Briefly, extracted protein (25 µL) was mixed with 200 µL BCA working reagent and incubated at 37° C. for 30 minutes. Absorbance was measured at 562 nm using a microplate reader (SpectraMax iD5, Molecular Devices).

### Double-Stranded DNA (dsDNA) Quantification

The amount of dsDNA in the native left ventricular tissue and in the ECM solution after decellularization was evaluated. Briefly, dsDNA was extracted using a standard DNA isolation kit (NuceloSpin, Macherey-Nagel, Düren, Germany) according to manufacturer’s instruction. The amount of extracted dsDNA was measured using a Nanodrop spectrophotometer (Thermo Fisher Scientific, Waltham, MA) at 260 nm wavelength. The tests were run in triplicate.

### Sodium Dodecyl Sulfate Polyacrylamide Gel Electrophoresis (SDS-PAGE)

Proteins extracted from ECM were loaded into 8-16 % sodium dodecyl sulfate-polyacrylamide gel (Bio-Rad, Cat. # 456-8104) and separated by electrophoresis. 15 µg of total ECM protein was loaded into each well of the polyacrylamide gel and compared to rat tail collagen type I (Corning, Cat. # 354236). The polyacrylamide gel was then stained with Imperial™ Protein Stain for visualization (Thermo Scientific, Prod. # 354236).

### Turbidimetric Gelation Kinetics

Turbidimetric gelation kinetics of ECM were performed and analyzed as described elsewhere (Johnson et al., Nanot., 22:494015 (2011)). 100 µL of 9, 12, and 20 mg/mL cold ECM solution (n=4) was loaded into a 96 well-plate in a microplate reader that was pre-heated to 37° C. The reading was recorded every 30 seconds for 90 minutes. The normalized absorbance (NA) was calculated according to Equation S1, where A is the absorbance at a given time, A0 is the absorbance at point 0, and Amax represents the maximum absorbance.

$$N A = \frac{A - {A}_{0}}{{A}_{m a x} - {A}_{0}}$$

For kinetic analysis, the time needed to reach 50 % Aₘₐₓ is defined as t_{½}; the lag phase, t_{lag}, was determined as the x-intercept by extrapolating the linear portion of the turbidimetric curve; and the slope of the curve was calculated as the speed of gelation, S.

### Dynamic Light Scattering

The hydrodynamic size of nanoclay (NC) was obtained using dynamic light scattering (DLS) (Wyatt Mobius). DLS was carried out with the NC dispersed in ultrapure water. Prior to DLS measurement, NCs were vortexed, sonicated, and then equilibrated for 5 minutes. DLS distribution is the average result of 3 independent samples with 9 repetitive measurements of each sample.

### Preparation of the Nanocomposite Hydrogels

ECM-NC gels were made by mixing neutralized ECM solution (20 mg/mL), 9 % (w/v) NC (Laponite^{®} XLG, BYK USA Inc., Rochester Hills, MI) and molecular biology grade water (Phenix Research Products, Candler, NC) at different weight ratios, shown in Table 1 and 2. Omnipaque (350 mgI/mL, GE Healthcare) was introduced into ECM-NC gels at 27 % w/w of iohexol for radiopacity, shown in Table 3. The homogenous mixing of ECM-NC gels was achieved by using a SpeedMixer (FlackTek Inc., Landrum, SC).

### Rheology

All rheological measurements were performed with a strain-controlled MCR 302 rheometer (Anton Paar USA Inc., Torrance, CA). A 25 mm diameter sandblasted aluminum upper disk and an aluminum lower plate were used, and the gap in between was kept at 1 mm for all measurements. In addition, a solvent trap was used, and the edge of the solvent trap was filled with water to provide a humidified environment to prevent drying.

For ECM solutions, large-amplitude oscillation sweep (LAOS) were performed at 10 rad/second. The gelation kinetics was examined using an isothermal test at a fixed strain of 0.5 % at 37° C. The shear rate sweeps of 20 mg/mL ECM solution were carried out at both 4° C. and 37° C. to assess its shear-thinning properties before and after gelation.

For ECM-NC gels, all rheological tests were performed at 37° C., unless otherwise denoted, following protocols described elsewhere (Avery et al., Sci. Transl. Med., 8(365):365ra156 (2016)). Shear rate sweeps performed to characterize the gel’s shear thinning behavior. LAOS were performed at both 25° C. and 37° C. at a fixed angular frequency of 10 rad/second. The above tests were run in triplicates. The yield stress was calculated from LAOS. Specifically, critical strain (εc) defined as the intersection of the segmented linear fittings on the stress-strain curve, was first measured. Yield stress, σy, was then extrapolated as the stress value corresponding to εc. During frequency sweeps, a strain range of 0.1 to 100 rad/second was scanned at a fixed strain of 0.5 % (in the linear viscoelastic region). Lastly, thixotropic test was conducted at 37° C. at 10 rad/second to evaluate time-dependent shear thinning property. The strain was oscillated between 100 % (for 1 minute) and 0.5 % (for 2 minutes) to examine the recoverability of the gels.

### Injectability

The injectability of ECM-NC gels through clinical catheters was investigated using a mechanical tester (Instron, Norwood, MA) as described elsewhere (Avery et al., Sci. Transl. Med., 8(365):365ra156 (2016)). The force required for ECM-NC gels (loaded into a 1 cc BD syringe) to pass a 2.8 F, 110 cm catheter (Terumo Medical Corporation, Somerset, NJ) at a flow rate of 1 mL/minute was recorded using Bluehill version 3 Software (Instron, Norwood, MA, US). Afterward, both break loose force and injection force were analyzed. Cell Culture: L-929 mouse fibroblasts (ATCC, Manassas, VA) were cultured at 37° C. in 5 % CO₂ atmosphere in the following medium: Eagle’s Minimum Essential Medium (ATCC, Cat. # 30-2003), 10 % Fetal Bovine Serum, and 1% Penicillin-Streptomycin.

### Cell Culture on xECM4.5NC Coated Plates

To assess cell viability in direct contact with xECM4.5NC, the gels were first spread on the bottom of 96-well plate by centrifugation at 1500 RPM for 3 minutes for complete coverage. L929 cells were seeded in at a density of 5000 each well directly on top of the gel and incubated at 37° C. overnight. The cell viability was accessed using CellTiter-Glo Luminescent assay (Promega, Cat. # G7572) according to the manufacturer’s instructions. After the cells were lysed, the top aliquot was carefully transferred into an opaque bottom plate, and the luminescent signal was read on a microplate reader immediately. The wells coated with gels but without cells were used as corresponding controls to each material to subtract the luminescent background from the readings. Three independent experiments were conducted with six replicates in each experiment.

### In Vitro Cytotoxicity

In vitro cytotoxicity evaluation of ECM, NC, EMH, and EMH-I were conducted according to ISO-10993-5. Briefly, 1 gram of each material was dissolved in complete cell culture medium and incubated at 37° C. for 24 hours. The supernatant and its series dilution (100 %, 50 %, 25 %, and 12.5 %) were prepared as treatment medium. In a 96-well plate, L-929 cells were seeded at a density of 5000 cells per well. After 24 hour incubation, the culture medium was aspirated and replaced with treatment medium (100 µL per well) for another 24 hours. Cell viability was analyzed using WST-1 reagents (Cayman Chemical, Ann Arbor, MI) according to the manufacturer’s protocol. Briefly, WST-1 solution was added to each well (10 µL), and the plate was incubated at 37° C. for 2 hours, followed by reading the absorbance at 450 nm. Dimethyl sulfoxide (DMSO) (10 %) was used as a positive control for cytotoxicity. Three independent experiments were conducted with four replicates in each experiment.

### Antibacterial Activity

The antibacterial activity of EMH and EMH-I was tested using Escherichia coli (E. coli) as described elsewhere with minor modifications (Han et al., Nanoscale, 11 :15846 (2019)). A 10 mLE. coli suspension with a concentration of 10⁷ CFU/mL was added on top of the 1 mL gel to reach a final concentration of 10⁸ CFU per milliliter gel. Gels with Luria-Bertani (LB) broth were used as negative controls. The groups were incubated for 24 hours at 37° C. at 180 rpm in a shaker incubator. The optical density of the suspension was measured at 600 nm using a microplate reader. Each suspension was measured three times, and each test was conducted three times independently.

### Fourier Transform Infrared Spectroscopy (FTIR)

The surface chemistry of the ECM, NC, EMH, and EMH-I was characterized using FTIR. FTIR spectra were acquired using an attenuated total internal reflectance Fourier transform infrared (ATR-FTIR) spectroscopy (Bruker TENSOR II with Platinum ATR Accessory). Each material was measured at least three times by randomly sampling from the bulk to ensure the consistency of the composition.

### Scanning Electron Microscopy (SEM)

A scanning electron microscopy (JCM-6000Plus) was used to visualize the microstructures of tissue samples before and after decellularization, ECM, and ECM-NC gels. For sample preparation, paraffin-embedded sections of native heart and decellularized heart (4 µm) were deparaffinized and air-dried. ECM solution was first gelled at 37° C. and then fixed with 4 % glutaraldehyde, followed by dehydration through a series of ethanol washes (started from 30 % ethanol and ended with 100 % ethanol) and critical point drying (Leica EM CPD300). NC, EMH, and EMH-I were first frozen at -80° C., followed by lyophilization (Labconco, 0.120 mBar, and -50° C.). All prepared specimens were then sputter-coated with 7 nm gold/palladium (Leica EM ACE200) and imaged using SEM.

### In Vitro Occlusion Model

The ability of NC, EMH, and EMH-I to withstand physiologically relevant pressure was examined using an in vitro occlusion model as described elsewhere (Avery et al., Sci. Transl. Med., 8(365):365ra156 (2016)). Briefly, PBS was pumped at 50 mL/minute using a syringe pump to displace the material inside of a tube. The maximum pressure that required displacing 1 mL of the material was recorded as the displacement pressure using a pressure sensor (Omega Engineering Inc., Norwalk, CT). Each test was conducted three times.

### In Vitro Retrieval Test

The retrievability of EMH-I was tested using a Penumbra System for aspiration (Penumbra, Alameda, CA). The retrieval process was monitored under fluoroscopy (OEC9800 plus C-Arm, GE Healthcare Systems, Chicago, IL).

### Rat Subcutaneous Injections

All animals used in this study were 4-5 week old Sprague Dawley rats (Charles River Laboratories, Wilmington, MA). 200 µL of saline (control), NC (4.5 wt %), EMH, or EMH-I were subcutaneously injected into lateral pockets of each rat under general anesthesia. The rats were sacrificed at day 3, day 14, and day 28 postimplantation, followed by tissue collection for histological examination.

### Arterial and Renal Embolization in a Porcine Model

The procedure was performed as described elsewhere (Avery et al., Sci. Transl. Med., 8(365):365ra156 (2016)). Healthy Yorkshire pigs (S&S Farms, Brentwood, CA) weighing 48 to 55 kg were acclimatized for at least 4 days under standard feeding conditions and suitable temperature. Before the embolization procedure, the pigs were first anesthetized using intramuscular injection of 5 mg/kg tiletamine-zolazepam (Telazol, Zoetis), 2 mg/mL xylazine, and 0.02 mg/kg glycopyrrolate. Following intubation, anesthesia was maintained with inhalation of 1.5-3 % isoflurane. During the procedure, percutaneous access to the carotid artery was obtained under the guidance of ultrasound (ACUSON S2000, Siemens) and fluoroscopy (OEC9800 plus C-Arm, GE Healthcare Systems, Chicago, IL). With a 5 French Bernstein catheter (Cook Medical) and a guidewire (GT- glidewire, Terumo Medical), angiography of the internal iliac (n=8) or renal artery (n=8) was performed under real-time fluoroscopic guidance using an intravenous contrast agent (350 mgI/mL Omnipaque, GE HealthCare, MA). EMH-I was delivered to the iliac or renal artery using a catheter. The radiopacity of EMH-I and vessel patency were assessed using fluoroscopy and digital subtraction angiography, respectively. Repeated angiography was performed to examine the embolic efficacy of EMH-I in vivo. Pigs were either sacrificed 1-hour post-embolization (non-survival group; n=4) or at 2 weeks post-embolization (survival group; n=4). In the survival group, hemostasis at the carotid arterial puncture site was achieved by manual compression and the wound was sealed using Dermabond (Ethicon, USA). Prior to euthanasia, blood samples were obtained for analysis, and whole-body CT was performed. At necropsy, the embolized internal iliac artery, or the kidneys were removed and examined by microCT and histopathology.

### Complete Blood Count (CBC) and Blood Biochemistry

CBC was carried out using an automatic analyzer (HemaTrue, Heska, Loveland, CO). CBC was measured to assess the hematological indices in rats and pigs, respectively, to monitor the overall animal health. In addition, blood biochemistry was also evaluated for pigs using a Veterinary Chemistry Analyzer (DRI-CHEM 4000, Heska, Loveland, CO).

### Whole Body CT Scans and Analysis

The pigs were scanned for embolized artery, and organs, as well as signs of distal migration of embolic agent (EMH-I) using whole-body CT performed on a clinical dual-source scanner (Siemens Force, Siemens Healthineers, Erlangen, Germany). During the scan, CT angiography (CTA) was performed by administrating contrast agent (Omnipaque, 350 mgI/mL, GE HealthCare, MA) intravascularly to visualized vasculature roadmap. The spiral scan was performed at 150 kVp and 80 kVp energy level, respectively, with a 0.6 mm detector size configuration. The segmentation and the volumes of the pig kidneys acquired from CT scans were analyzed using Visage 7.1 (Visage Imaging Inc., San Diego, California).

### Hematoxylin and Eosin (H&E) Staining

H&E staining (Thermo Fisher Scientific, Cat. # 7111 and 7221, Waltham, MA) was performed on paraffin-embedded sections of cardiac tissues (before and after decellularization), rat subcutaneous tissues, pig vessels, and pig kidneys.

### Masson’s Trichrome Staining

Masson’s trichrome staining (Thermo Fisher Scientific, Cat. # 22-110-648, Waltham, MA) was performed on paraffin-embedded sections of rat subcutaneous tissues and pig iliac arteries to detect connective and muscle tissues.

### Elastic Stain

Elastic histochemical stain staining (Sigma Aldrich, Cat. # HT25A, St. Louis, MO) was performed according to manufacturer’s instructions to identify the internal elastic lamina in explanted pig vessels.

### Immunohistochemistry (IHC)

Immunohistochemical staining for collagen-I, fibronectin, and laminin was performed on cardiac tissue and on EMH-I embolized internal iliac artery at D0 to visualize the presence of extracellular matrix components. For rat subcutaneous injections, myeloperoxidase (MPO) and CD31 were stained. For pig vessels, MPO, and proliferating cell nuclear antigen (PCNA) immunohistochemistry staining was performed as described elsewhere (Avery et al., Sci. Transl. Med., 8(365):365ra156 (2016)). Briefly, paraffin-embedded sections underwent deparaffinization, endogenous peroxidase quenching, antigen retrieval, and then incubated with 5 % (v/v) goat serum blocking solution (in 1X PBS) for 1 hour at room temperature. For fluorescence IHC, the sections were stained with following antibodies at 4° C. overnight: rabbit polyclonal to Collagen I (Abcam, ab34710, 1:500), mouse monoclonal (IST-9) to Fibronectin (Abcam, ab6328, 1:100), and rabbit polyclonal to Laminin (Abcam, ab11575, 1:200). Alexa Fluor 594 goat anti-mouse IgG (Invitrogen, Cat. # R37121) and Alexa Fluor 594 goat anti-rabbit IgG (Invitrogen, Cat. # A-11037) were used as secondary antibodies. Coverslips were mounted with Antifade Mounting Medium with DAPI (Vectashield, Cat. # H-1200) and imaged using an EVOS FL Auto 2 Imaging System (Thermo Scientific Invitrogen). For colorimetric IHC, the sections were stained with following antibodies at 4° C. overnight: anti-myeloperoxidase antibody (Abcam, ab208670, 1:200), recombinant anti-CD31 antibody (Abcam, ab182981, 1:200), recombinant anti-PCNA antibody (Abcam, ab92552, 1:200). Goat anti-rabbit IgG H&L (HRP) (Abcam, ab97051, 1:200) was used as the secondary antibody for 1-hour incubation as room temperature. 3′-Diaminobenzidine substrate (Vector Laboratories, SK-4100) was used for color development, which was monitored under a light microscope. Tissue sections were then counterstained with hematoxylin, dehydrated, mounted, and imaged. Slides with no primary antibodies were included as controls for all samples to confirm the specificity of primary antibodies.

### MicroCT Imaging and Analysis

Excised pig iliac arteries and kidneys were scanned with a microCT (Skyscan 1276, Bruker Corporation, Kontich, Belgium). The pig iliac arteries were scanned using a current of 200 µA and a voltage of 45 kV with a 0.25 mm aluminum filter at 20 µm resolution and 0.4° rotational step. Harvested pig kidney samples were scanned using a current of 200 µA and a voltage of 55 kV with a 0.5 mm aluminum filter at 80 µm resolution and 0.8° rotational step. The microCT images were then reconstructed using NRcon reconstruction software (Bruker Corporation, Kontich, Belgium) for further analysis.

To acquire the volumes of the embolized EMH-I within the iliac artery, the reconstructed microCT images were loaded into the segmentation software Mimics (Materialise, Leuven Belgium). The EMH-I and connective tissue were segmented based on densities by thresholding. The 3D model of EMH-I was reconstructed, and the volume was generated using 3-Matics (Materialise, Leuven Belgium).

### Statistical Analysis

Statistical analysis was performed with PRISM 8 (GraphPad Software, San Diego, CA). One-way analysis of variance (ANOVA) with a multiple comparison method was performed for experiments containing more than two groups. Two-way ANOVA analysis followed by Tukey’s multiple comparison test was performed For comparison between multiple groups at multiple time points. The two-tailed, unpaired t-test was performed for experiments with two groups. p < 0.05 was defined as statistically significant.

### OTHER EMBODIMENTS

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

1. A hydrogel composition comprising decellularized extracellular matrix (ECM) and a nanoclay material.

2. The hydrogel composition of claim 1, wherein said hydrogel composition comprises about 1 wt% of said decellularized ECM.

3. The hydrogel composition of claim 1, wherein said hydrogel composition comprises from about 1 wt% to about 5.5 wt% of said nanoclay material.

4. The hydrogel composition of claim 3, wherein said hydrogel composition comprises about 4.5 wt% of said nanoclay material.

5. The hydrogel composition of claim 1, wherein said nanoclay material is a silicate nanoclay.

6. The hydrogel composition of claim 1, said hydrogel composition further comprising a radiopaque contrast agent.

7. The hydrogel composition of claim 6, wherein said hydrogel composition comprises from about 18 wt% to about 27 wt% radiopaque contrast agent.

8. The hydrogel composition of claim 7, wherein said hydrogel composition comprises from about 27 wt% of said radiopaque contrast agent.

9. The hydrogel composition of claim 1, wherein said radiopaque contrast agent is selected from the group consisting of iohexol, tantalum microparticles, iodized oil, and iodixanol.

10-15. (canceled)

16. A method for treating a mammal having a bleeding disorder, wherein said method comprises administering, to said mammal, a hydrogel composition comprising decellularized ECM and a nanoclay material.

17. The method of claim 16, wherein said bleeding disorder is selected form the group consisting of a non-traumatic hemorrhage, a traumatic hemorrhage, a ruptured aneurysm, a saccular aneurysm, a vascular malformation, and an endoleak.

18. A method for treating a mammal having a tumor, wherein said method comprises administering, to a blood vessel within said mammal that is feeding said tumor, a hydrogel composition comprising decellularized ECM and a nanoclay material.

19. The method of claim 18, wherein said tumor is a benign tumor.

20. The method of claim 18, wherein said tumor is a malignant tumor.

21. The method of claim 18, wherein said tumor is selected from the group consisting of hepatic tumors, uterine fibroids, and prostate tumors.

22. The method of claim 18, wherein said mammal is a human.

23. The method of claim 18, wherein said administering comprises catheter-directed administration.

24. The method of claim 18, wherein said administering comprises administering from about 1 cc to about 3 cc of said hydrogel composition.

25. The method of claim 16, wherein said mammal is a human.

26. The method of claim 16, wherein said administering comprises catheter-directed administration.
